SEQUENCE LISTING (10 sequences)

SEQ ID NO: 1 - DNA, 7478 bp, Hepatitis A virus
ttcaagaggggtctccgggaatttccggagtccctcttggaagtccatggtgaggggacttgatacctcaccgccgtttgcctaggctataggctaaattttccctttcccttttccctttcctattccctttgttttgcttgtaaatattaattcctgcaggttcagggttcttaaatctgtttctctataagaacactcatttttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcagggctgtctttaggcttaattccagacctctctgtgcttagggcaaacatcatttggccttaaatgggattctgtgagaggggatccctccattgacagctggactgttctttggggccttatgtggtgtttgcctctgaggtactcaggggcatttaggtttttcctcattcttaaataataatgaacatgtctagacaaggtattttccagactgttgggagtggtcttgaccacatcctgtctttggcagacattgaggaagagcaaatgattcaatcagttgataggactgcagtgactggtgcttcttattttacttctgtggatcaatcttcagttcatacagctgaggttggatcacaccaggttgaacctttgagaacctctgttgataaacccggttcaaagaagactcagggagagaaatttttcttgattcattctgcagattggcttactacacatgctcttttccatgaagttgcaaaattggatgtggtgaaattattatacaatgagcagtttgctgttcaagggttgttgagataccatacatatgcaagatttggcattgaaattcaagttcagataaaccctacacctttccaacaggggggattgatctgtgctatggttcctggtgaccagagctatggttctatagcatcattgactgtttatcctcatggtttgttaaattgcaatattaacaatgtggttagaataaaggttccatttatttacacaagaggtgcttaccactttaaagatccacaatacccagtttgggaattgacaattagagtttggtcagaattaaatattgggacaggaacttcagcttatacttcactcaatgttttagctagatttacagatttggagttgcatggattaactcctctttctacacaaatgatgagaaatgaatttagggtcagtactactgagaatgtggtgaatctgtcaaattatgaagatgcaagagcaaagatgtcttttgctttggatcaggaagattggaaatctgatccgtcccagggtggtgggatcaaaattactcattttactacttggacatctattccaactttggctgctcagtttccatttaatgcttcagactcagttggtcaacaaattaaagttattccagttgacccatattttttccaaatgacaaatacgaatcctgaccaaaaatgtataactgctttggcttctatttgtcagatgttttgtttttggagaggagatcttgtctttgattttcaagtttttcccaccaaatatcattcaggtagattactgttttgttttgttcctggcaatgagctaatagatgtttctggaatcacattaaagcaagcaactactgctccttgtgcagtaatggatattacaggagtgcagtcaactttgagatttcgtgttccctggatttctgacactccttacagagtgaacaggtatacaaagtcagcacatcagaaaggtgagtacactgccattgggaagcttattgtgtattgttataacagattgacctctccttctaacgttgcttcccatgtcagagtgaatgtttatctttcagcaattaacttggaatgttttgctcctctttatcatgctatggatgttactacacaagttggagatgattctggaggtttttcaacaacagtttctacagaacagaatgttccagatccccaagttggtataacaaccatgaaagatttgaaaggaaaagctaacagagggaaaatggatgtttcaggagtacaagcacctgtgggagctatcacaacaattgaggatccagttttagcaaagaaagtacctgagacatttcctgaattgaaacctggagaatccagacatacatcagatcatatgtccatctacaagtttatgggaaggtctcatttcttgtgcacttttacattcaattcaaataataaagagtacacatttcctataaccttgtcttcaacctctaatcctcctcatggtttgccatcaacactgaggtggtttttcaacttgtttcagttgtatagagggcctttagatctgacaattattattacaggagcaactgatgtagatggcatggcctggttcactccagtaggtcttgccgttgatactccttgggtagagaaggagtcagctttgtctattgactacaaaactgctcttggagctgtcagatttaacacaaggagaacagggaacattcagattagattaccatggtattcttatttatatgctgtgtctggagcactggatggtttgggtgacaagacagattctacatttggattggtttctattcagattgcaaattacaatcattctgatgaatacttgtcttttagttgttatttgtctgtcacagaacaatcagagttttattttcccagagctccattgaactcaaatgccatgttatccactgaatcaatgatgagcagaattgcagctggagacttggagtcatcagtggatgatcctagatcagaggaagataaaagatttgagagtcatatagaatgcaggaagccatataaagaactgagattagaagttgggaaacaaagactcaagtatgctcaggaagaattgtcaaatgaagtacttccaccccctaggaaaatgaagggactgttttcacaagccaaaatttctcttttttatactgaggagcatgaaataatgaagttttcctggagaggtgtgactgctgatactagagctttaaggaggtttggattctctttggccgcaggcagaagtgtgtggactcttgaaatggatgctggggttcttactgggagactgattagattgaatgatgagaaatggacagaaatgaaggatgacaagattgtttcattgattgaaaagtttacaagtaacaaatattggtccaaagtgaatttcccacatgggatgttggatcttgaagaaattgctgccaattctaaggattttcctaacatgtctgaaacggatttgtgtttcttgctgcattggttaaatccaaagaaaattaatttagcagatagaatgcttggattgtctggagttcaggaaattaaagaacaaggtgttggattaatagcagagtgtagaactttcttagattctattgctggaactttaaaatctatgatgtttggatttcatcattctgtgactgttgaaattataaacactgtgctctgttttgttaagagtggaattttgctttatgtaatacaacaattgaatcaggatgaacattctcacataattggtttgttgagagtcatgaattatgcagatattggttgttcagttatttcatgtggcaaagttttttccaaaatgctggaaacagtctttaattggcaaatggactccagaatgatggagttaaggactcagagtttttccaactggttaagagatatttgttctgggatcaccatttttaaaaacttcaaggatgcaatttattggctttatacaaaattaaaggacttttatgaagtgaattatggcaagaagaaggacattttaaatattcttaaagataaccaacaaaaaatagagaaagccattgaggaagccgatgaattttgcattttgcaaatccaagatgtggaaaaatttgaacagtatcagaaaggggttgacttgatacaaaaattgagaactgttcattcaatggctcaggttgatccaaatttaatggttcatttgtcacctttgagagattgtatagcaagagttcatcagaaacttaaaaaccttggatctataaatcaggcaatggtaacgagatgtgagccagttgtttgttatttatatggcaaaagagggggaggaaagagcttaacatcaattgcattggcaaccaaaatttgtaaacattatggtgttgagcctgaaaagaatatctatactaaacctgtggcttcagattactgggatggatatagtggacaattagtttgcatcattgatgatattggccaaaacacaacagatgaggattggtcagatttttgtcagttagtgtcaggatgtccaatgagattaaacatggcctctcttgaggagaagggtaggcatttttcttctccttttataatagcaacttcaaattggtcaaatccaagtccaaaaacagtttatgttaaggaagcaattgaccgcagactccatttcaaggttgaagttaaacctgcttcatttttcaaaaatcctcacaatgatatgttgaatgttaatttagctaaaacaaatgatgcaatcaaagatatgtcttgtgttgatttgataatggatggacataatgtttcattgatggatttgctcagttctttagtcatgacagttgaaattagaaaacaaaacatgactgaattcatggagttgtggtctcagggaatttcagatgatgataatgatagtgcagtagctgagtttttccagtcttttccatctggtgaaccatcgaactctaaattatctggctttttccaatctgttactaatcacaagtgggttgctgtgggagctgcagttggcattcttggagtgctcgttggaggatggtttgtgtataagcatttctcccgcaaagaggaggaaccaatcccagctgaaggggtatatcatggtgtaactaagcccaagcaagtgattaaattagatgcagatccagtagaatctcagtcaactttggaaatagcaggactggttaggaagaacttggttcagtttggagttggagagaagaatggatgtgtgagatgggttatgaatgccttgggagtgaaagatgattggctgcttgtgccttcccatgcttataaatttgagaaagattatgaaatgatggagttttattttaatagaggtggaacttactattcaatttcagctggtaatgttgttattcaatctttggatgtgggattccaggatgttgttctgatgaaggttcctacaattcctaagtttagagatattactcagcattttattaagaaaggggatgtgcctagagctttgaatcgcctggcaacattagtgacaactgtaaatggaacccctatgttaatttctgagggcccactaaagatggaagagaaagctacttatgttcataagaaaaatgatggtacaacagttgatttaactgtggatcaggcatggagaggaaaaggcgaaggtcttcctggaatgtgtggtggggccttggtttcatcgaatcaatctatacagaatgcaatcttgggcatccatgttgctggaggaaattcaattcttgttgcaaaattggttactcaagaaatgttccaaaatattgataagaaaattgaaagtcagagaattatgaaagtggagtttactcagtgttcaatgaatgtggtctccaaaacgctttttagaaagagtcccatttatcatcacattgataaaaccatgattaattttcctgcagctatgcccttttctaaagctgaaattgatccaatggctgtgatgttatctaagtattcattacctattgtagaagaaccagaggattataaagaggcttcaattttttatcaaaataaaatagtgggtaagactcagttagttgatgattttttagatcttgatatggccattacaggggccccaggaattgatgctatcaacatggattcatctcctggatttccttatgtccaggagaagttgaccaaaagagatttaatttggttggatgaaaatggtttattgctgggagttcatccaagattggctcagagaatcttattcaatactgtcatgatggaaaattgttctgatttggatgttgtttttacaacctgtccaaaagatgaattgagaccattagagaaagtgttggaatcaaaaacaagagctattgatgcttgtcctctggattactcaattttgtgccgaatgtattggggtccagctattagttattttcatttgaatccaggtttccatacaggtgttgctattggcatagatcctgatagacagtgggatgaattatttaaaacaatgataagattcggagatgttggtcttgatttagatttctctgcttttgatgctagtcttagtccatttatgattagagaagcaggtagaatcatgagtgaactatctggaactccatcccattttggcacagctcttatcaatactatcatttattccaagcatttgctgtataactgttgttaccatgtctgtggttcaatgccctctgggtctccttgtacagctttgctaaattcaattattaataatgtcaatttgtattatgtgttttccaagatatttggaaagtctccagttttcttttgtcaggctttgaagattctctgttatggagatgatgttttaatagttttctctcgagatgttcagattgataatcttgatttgattggacaaaaaattgtagatgagtttaagaaacttggcatgacagctacttctgctgacaagaatgtacctcagctgaaaccagtttcggaattgacttttctcaaaagatctttcaatttggtagaggatagaattagacctgcaatttcggaaaaaacaatttggtctttaatagcatggcagagaagtaacgctgagtttgagcagaatttagaaaatgctcagtggtttgcttttatgcatggctatgagttttatcagaaattttattattttgttcagtcctgtttggagaaagagatgatagaatacagacttaaatcttatgattggtggagaatgagattttatgaccagtgtttcatttgtgacctttcatgatttgtttaaacaaattttcttaaaatttctgaggtttgtttatttcttttatcagtaaat

SEQ ID NO: 2 - DNA, 32 bp, Artificial Sequence
  PCR forward primer A
gttttattttcccagagctccattgaactcaa

SEQ ID NO: 3 - DNA, 72 bp, Artificial Sequence
  PCR reverse primer B
ggtacctacgtagtcgactccgccacctctagaattggcttgtgaaaacagtcccttcttcattttcctagg

SEQ ID NO: 4 - DNA, 72 bp, Artificial Sequence
  PCR primer C
gactacgtaggtaccgggggaggcggatccctgttttcacaagccaatatttctcttttttatactgaggag

SEQ ID NO: 5 - DNA, 36 bp, Artificial Sequence
  PCR primer D
atttttccacatcttggatttgcaaaatgcaaaatt

SEQ ID NO: 6 - DNA, 36 bp, Artificial Sequence
  Synthetic oligonucleotide primer
gtcgacgtcgaccaggccaagcctttgtctcaagaa

SEQ ID NO: 7 - DNA, 36 bp, Artificial Sequence
  Synthetic oligonucleotide primer
cggttaggtaccgccctcccacacataaccagaggg

SEQ ID NO: 8 - DNA, 21 bp, Artificial Sequence
  PCR forward primer A1
gagtcatgaattatgcagata

SEQ ID NO: 9 - DNA, 21 bp, Artificial Sequence
  PCR reverse primer A2
aaccaatatctgcataattca

SEQ ID NO: 10 - DNA, 72 bp, Artificial Sequence
  Plasmid pHAV8Y-MCS derived from Hepatitis A virus
caagccaattctagaggtggcggagtcgactacgtaggtaccgggggaggcggatccctgttttcacaagcc

(19) United States
(12) Patent Application Publication
(10) Pub. No.: US 2010/0055670 A1
(43) Pub. Date: Mar. 4, 2010

(54) GROWTH OF WILD-TYPE HEPATITIS A VIRUS IN CELL CULTURE

(75) Inventors: Gerardo Kaplan, Bethesda, MD (US); Krishnamurthy Konduru, Bethesda, MD (US)

Correspondence Address: BIRCH STEWART KOLASCH & BIRCH, PO BOX 747, FALLS CHURCH, VA, 22040-0747, US

(73) Assignee: The Government of the US, Represented by the Secretary, Dept. of Health and Human Services, Rockville, MD (US)

(21) Appl. No.: 11/993,851

(22) PCT Filed: Jun. 26, 2006
(86) PCT No.: PCT/US06/24748

§ 371 Date: Jun. 24, 2008

Related U.S. Application Data
(60) Provisional application No. 60/694,232, filed on Jun. 28, 2005.

(51) Int. Cl. C12Q 1/70; C07H 21/04; C12N 15/63; C12N 5/071; C12N 7/02; C12P 19/34

(52) U.S. Cl. 435/5; 536/23.72; 435/320.1; 435/325; 435/370; 435/239; 435/91.1

(57) ABSTRACT

The invention provides recombinant Hepatitis A Virus (HAV) nucleic acids and host cells that are permissive for their growth and replication. The recombinant Hepatitis A Virus nucleic acids not particularly limited, except that they incorporate at least one heterologous nucleic acid fragment. The heterologous nucleic acid can encode a selectable marker gene and such recombinant HAV nucleic acids are useful for selecting cells that are permissive for growth and replication of wild type HAV. Alternatively, the heterologous nucleic acid may encode a vaccine antigen or other expression product that is desirable to express in a cell harboring the recombinant HAV nucleic acid. The invention further provides cell lines permissive for growth and replication of wild type HAV or HAV having minimal mutations for growth in cell culture. The invention further provides methods for producing HAV vaccines and for monitoring environmental and patient samples for the presence of HAV.

### U.S. GOVERNMENT INTEREST IN THE INVENTION

The present invention was made, at least in part, with funding provided by the United States Government as represented by the Department of Health and Human Services. Accordingly, the United States Government may have certain rights in the invention.

### FIELD OF THE INVENTION

The present invention relates to recombinant Hepatitis A Virus (HAV). The invention encompasses recombinant HAV genomes and assembled virus particles, these being useful as vaccines and also as vectors for introducing the recombinant HAV genomes they contain into cells for various purposes. The invention further relates to cells and cell lines that can be used to grow wild-type and altered HAV viruses in culture for various purposes, including diagnostic and environmental monitoring purposes.

### BACKGROUND OF THE INVENTION

Hepatitis A virus (HAV) is a Picornavirus that causes acute hepatitis in humans, a preventable infectious disease that is nevertheless prevalent worldwide. In the United States, approximately 25,000 cases of HAV are reported each year, however an estimated average of 263,000 HAV cases occur annually when corrected for underreporting and asymptomatic infections (16).

HAV is a non-enveloped virus that contains a 7.5 kb single-stranded positive-sense genomic RNA encapsidated in an icosahedral 27-32 nanometer (nm) diameter particle. A small virus-encoded protein (VPg) is covalently linked to the 5′ end of the genome. The viral RNA contains at the 5′-end a nontranslated region (“5′-NTR” or “5′-NC”) of approximately 750 bases with an internal ribosome entry site (IRES) (42), and at the 3′-end a short nontranslated region followed by a poly(A) tail. There are two in-frame start (AUG) codons, one at nucleotides 735-737 and the other at nucleotides 741-743 nt; both are located downstream from the IRES. Translation of a large open reading frame that codes for a polyprotein of about 250 kDa usually starts the second AUG (37). The virus encoded protease 3 Cpro cleaves the HAV polyprotein into smaller structural (VP0, VP3, VP1-2A) and nonstructural (2B, 2C, 3A, 3B, 3C, and 3D) proteins (22, 25, 31 and papers cited in reference 31). Unlike other picornaviruses, a cellular protease cleaves the VP1-2A precursor (21).

The HAV encoded protease from the nonstructural 3C gene cleaves viral proteins by a process occurring simultaneously with translation and having a posttranslational aspect (2, 36). VP4 protein is a first translated polypeptide of 21-23 amino acids with a maximum molecular mass of 2.5 kD that has not yet been found in the HAV viral capsid. In other picornaviruses, the VP4 protein is slightly larger in size (about 7 kD) and myristylated and may be involved in particle assembly, stability or viability and also in cell binding and entry of the virus into cells (27, 37, 38).

### BRIEF DESCRIPTION OF THE DRAWINGS

FIG. 1. Schematic representation of HAV vector constructs. A polylinker including SalI, SnaBI, and KpnI restriction sites, flanked by HAV protease 3 Cpro cleavage sites and Gly hinge sequences, was cloned at the 2A/2B junction of the HAV cDNA in pHAV8Y (SEQ ID NO: 10). The resulting plasmid is named pHAV8Y-MCS. A blasticidin-resistance (bsd) gene without translation and initiation codons was cloned into the SalI and KpnI sites of pHAV8Y-MCS. The resultant construct, termed pHAV8Y-Bsd, encodes the bsd gene in-frame with the polyprotein and its gene product Bsd should be released from the polyprotein by 3C^{pro} cleavage.

FIG. 2. Immunofluorescence (IF) analysis of wild-type (wt) HAV rescued from transfected Huh7 cells. Mock-transfected Huh7 cells (A and D) and blasticidin-resistant Huh7 cells transfected with HAV8Y-Bsd (B and E) or HAV.WT-Bsd (C and F) in vitro synthesized RNA and selected with 1 μg/ml blasticidin were grown in 8-well slides. The cells were fixed with cold acetone, and stained with anti-HAV Mabs K2-4F2 and K3-4C8 (Commonwealth Serum Laboratories, Melbourne, Australia) and FITC-conjugated goat anti-mouse antibodies. FRhK-4 cells were infected with viral stocks from mock-, HAV8Y-Bsd-, and HAV.WT-Bsd-transfected Huh7 cells for 2 weeks, fixed with cold acetone, and stained with anti-HAV Mabs K2-4F2 and K3-4C8 and FITC-conjugated goat anti-mouse antibodies. Immunofluorescent micrographs were taken with a Zeiss Axioscope microscope at 400× magnification using an oil immersion objective.

FIG. 3. Stability of wt HAV in Huh7 cells. Titers of HAV8Y-Bsd and HAV.WT-Bsd grown in Huh7 cells were assessed in Huh7 and FRhK-4 cells using a blasticidin-resistance endpoint titration assay. Ten-fold dilutions of the viral stocks were titrated in 96-well plates containing subconfluent monolayers of Huh7 or FRhK-4 cells and, after over-night incubation, blasticidin was added to each well to 2 μg/ml. Eight wells/dilution were used in the titration assay. Plates were observed under the microscope 7 days after infection, and wells containing surviving cells were counted as positive for the titer determination. Values are log 10 of the HAV titers determined by the Reed and Muench method (34) and the standard deviations are shown as lines.

FIG. 4. Huh7 cells cured from HAV8Y-Bsd infection.

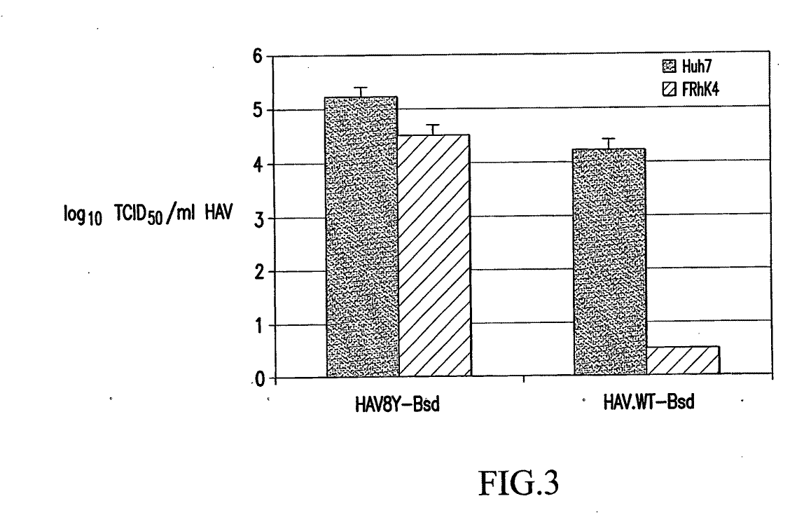

A) IF analysis. Uninfected Huh7 cells (mock), HAV8Y-Bsd-infected Huh7 cells (HAV8Y-Bsd), and the HAV8Y-Bsd-infected cells cured by treatment with 250 U/ml IFN-αA/D (cured), were grown in 8-well chamber slides and stained with anti-HAV monoclonal antibodies K2-4F2 and K3-2F2 for IF analysis. B) Growth of HAV8Y-Bsd and HAV.WT-Bsd in naïve Huh7 cells and interferon-cured Huh7-A-I cells. Viral titers were assessed in Huh7-A-I cells using the blasticidin-resistance endpoint titration assay (see legend FIG. 3). C) Growth of wt HM-175 HAV derived from human stools in naïve Huh7 cells and interferon-cured Huh7-A-I cells. Viral titers were assessed in Huh7-A-I cells using the ELISA endpoint titration assay. Values are log 10 of the HAV titers determined by the Reed and Muench method (34) and the standard deviations are shown as lines.

FIG. 5. Growth of wt HAV in different cell lines. One-step growth curve analysis of growth of various strains of HAV was performed in three different cell lines, FRhK4, Huh7 and Huh7-A-I cells (Huh7 cells that had been selected following infection with HAV8Y-Bsd and then cured by interferon treatment). Cells were infected with wild-type HAV recombinant viruses containing the Bsd selectable marker (HAV8Y-Bsd and HAV.WT-Bsd), wt HAV isolated from human stools (wt HM-175 HAV), or cell culture-adapted HAV (HAV/7), and viral growth was determined at different time points. Viral titers were assessed in Huh7-A-I cells using the ELISA endpoint titration assay. At each timepoint, samples are taken from infected FrhK-4, Huh7, and Huh7-A-I cells and titrated by ELISA in Huh7-A-I cells. Values are log 10 of the HAV titers determined by the Reed and Muench method (34) and the standard deviations are shown as lines.

FIG. 6. Schematic representation of the nucleotide sequence analysis of HAV genomic RNA extracted from the last point of the growth curve. The nucleotide sequence of parts of the virus obtained at the last time point of the one-step growth curves was analysed. Nucleotide sequences of the 5′ NTR and 2B-2C region hot-spots (black bars) of HAV8Y-Bsd, HAV.WT-Bsd, wt HM-175 HAV, and HAV/7 were obtained. Nucleotides that differ from the wt HM-175 HAV are indicated with the nucleotide position under the schematic representation of each the viral genomes. The main cell culture-adapting mutation at nt 3889 is indicated in bold. Viral genes, the internal ribosomal entry site (IRES), and the 3′ end poly(A) tail are indicated. The bsd gene clone in HAV8Y-Bsd and HAV.WT-Bsd is shown as a grey box.

### DESCRIPTION OF THE INVENTION

Among picornaviruses, HAV replicates inefficiently in cell culture. HAV has been adapted via passage in culture to grow in variety of primate cell lines and establishes persistent infection. However it does not cause a cytopathic effect in culture. Even though HAV adapts in some cells, generally it is difficult to adapt and grow in tissue culture conditions. Serial passage will provide HAV variants that will grow in cell culture, however the growth tends to be restricted to specific HAV strain and cell type combinations. It has been documented that some primary and continuous primate cell lines like fetal rhesus monkey kidney (FRhK4), African green monkey kidney (AGMK), human diploid lung (MRC5), and BSC-1 cells support HAV growth in cell culture (10, 43-45). The cell culture-adapted HAV usually takes many days to reach titers of virus of 10⁶-10⁷ (23). However, the rates of virus growth vary depending upon the combination of mutations that are present in the HAV genome and the host cell employed.

Sequence comparisons between wild-type (wt) HAV strain HM175 and its cell culture-adapted variant HAV/7, and many studies of their chimeras identified that the capacity of efficient replication is can be achieved through acquired mutations in the HAV genome (6, 11, 12, 14, 15, 46). Cell culture-adapted HAV isolates include several mutations throughout the viral genome are apparently involved in efficient growth of HAV in cell culture (11, 12, 14, 15, 19, 20, 40, 46-49). Two main hot-spots for culture-adapted mutations are found; one located in the 5′-NTR IRES and another located within nonstructural 2B and 2C genes encoding viral RNA replication proteins. These two hot-spot mutations are associated with efficient HAV replication in cell culture (3, 24). Mutations identified from chimeras of HAV/7 suggest that the HAV non-structural 2B and 2C coding regions are essential for virus growth in cell culture (12). Mutations in regions other than 2B and 2C did not have independent effect; however combining mutations in other parts together with 2B and 2C coding regions has heightened replication (13). Similarly the replication enhancement was attained in cell culture with mutations in the 5′-NTR region together with the 2B and 2C coding regions, whereas mutation only in the 5′NTR had no autonomous effect (3, 20, 40).

It has been suggested that mutations in the region of the 2B and 2C genes of the HAV genome, at nucleotides 3889, 4087 and 4222, are the minimal set needed to provide for growth in “permissive” cell lines such as FRhK4 and AGMK cells. These mutations are sufficient to provide growth in culture. These mutations are effective in any combination of two, but only the 3889 mutation appears to be effective alone (14).

Mutations in the 5′NTR portion of the viral genome provide for broadening of host cell range, allowing growth in less permissive cell lines. One set of 5′-NTR mutations, at nucleotides 124, 131 to 134, 152 and 203, is found to increase the rate of viral growth in BS-C-1 cells. These mutations do not affect the growth rate of HAV in permissive cells. A second set of mutations is found at nucleotides 591, 646, 669, 687 and independently increases the rate of replication in MRC5 cells (50). It has been shown that a specific mutation at the hot-spot nucleotide 3889, changing the 2B protein amino acid 216 from Ala (wild-type) to Val (8Y) has a major impact on viral replication in cell culture, providing a 10- to 20-fold increase in efficiency of replication in FrHK4 sub-line 11-1 cells. However, HAV bearing either the wild-type Ala or the mutant Val replicated with similar efficiency in vivo in chimpanzee and tamarin animals (18).

Although HAV expression vectors have been developed (1, 41), strains of HAV carrying antibiotic resistance genes that could allow the selection of infected cells have not been described. The examples herein provide a recombinant HAV genome having a blasticidin antibiotic resistant gene cloned into the 2A-2B junction of HAV that can be used to identify and select cells capable of supporting the efficient growth of human wt HAV in cell culture. The availability of these cell lines allows the isolation of wt HAV strains from the environment. This allows monitoring of food and water for the presence of infectious wt HAV. The ability to culture wild-type HAV from patient samples will facilitate the diagnosis of wt HAV infections. Moreover, the present invention is useful for the identification of cellular factors required for the growth of wt HAV as well as determinants of hepatovirulence and pathogenesis of HAV, and the development of HAV strains that could be used as attenuated vaccines for humans.

Currently available cell culture adapted strains of HAV are overly attenuated for humans, and cannot be used as an HAV vaccine because the virus does not replicate in vaccinees. The cell culture adapted HAV grows adequately in cell culture and can be used as a source of HAV antigen for killed vaccines. Since before this invention it was not possible to grow wild-type human HAV in cell culture, it was extremely difficult to introducing attenuating mutations in the virus that could lead to the development of a strain of HAV with the correct level of attenuation to be used as a vaccine candidate. One way to make an immunogenic, attenuated HAV variant would be to start with a wild-type or other highly immunogenic HAV isolate. Then one would introduce the mutations that attenuate the pathology of the virus in vivo, for example by reducing its replication rate in the tissues of the animal or changing the tropism of the virus to another organ or tissue. Unfortunately, there is at present no clear knowledge of such attenuating mutations, and so some method for identifying them is needed. That is, it is desirable to develop a system for culturing wild-type HAV, so that mutations that attenuate pathology, while preserving immunogenicity, can be introduced and investigated. The present invention provides such a system.

Furthermore, it has generally been the case that cell lines that are more “permissive” for growth of lesser attenuated strains provide for even faster growth of more attenuated strains. For example, MRC5 cells, the cell line licensed for production of present HAV vaccines, are considered to be a moderately permissive cell line. On the other hand, BS-C-1 cells are considered to be a more permissive cell line. The titer of attenuated HAV viruses that can be obtained is typically from 0.5 to 1 log unit higher in BS-C-1 cells than in MRC5 cells (50). Therefore, we consider that a cell line that supports growth of wild-type HAV, which should constitute a most permissive cell line, will be of great value as a cell line for production of HAV vaccines by allowing growth of the vaccine strain to very high titer. Indeed, the cured cell lines described herein support attenuated HAV titers at least as high as 10⁷ per TCID₅₀/ml (FIG. 5). Most importantly, the cell lines of the present invention support growth of HAV without accumulation of mutations during the culturing. Titers of wt HAV typically reach at least 10⁵ TCID₅₀ after 16 days in culture in the cured cell lines of the invention and furthermore, these titers are reached without the accumulation of mutations in the viral genome. Accumulation of mutations during culture as is typical for cell lines presently used to grow HAV in culture may overly attenuate a virus as to replication in human, rendering the virus useless as a live vaccine.

Furthermore, the rate at which wild-type HAV grows in typical cell lines of the present invention is almost as rapid as the rate of growth of culture-adapted strains, and for example HAV8Y. The virus titer of wild-type HAV in typical cells of the invention will increase by from 0.5 to 1.25 log units TCID₅₀ every four days once infection is established. Cell lines of the present invention preferably support a growth rate of wild type HAV of at least 0.9, more preferably at least 1 log unit TCID₅₀ over four days. The rate of growth of virus is typically stable up to at least 16 days, and preferably is stable indefinitely. It is noted that cells used for growing HAV are maintained in a proliferating state by splitting the culture periodically; typically at a 1:5 or 1:10 ratio once per week.

The rate of growth of cell culture-adapted HAV strains is typically even higher than the growth rate of wild-type virus. Thus, the virus titer of a culture-adapted strain of HAV may be as high as 1.5 log units TCID₅₀ over four days, or even higher. Again, such titers are obtained without the accumulation of additional attenuating mutations (that is, in addition to those that provide the original culture adaptation) in the HAV genome.

In spite of great progress that has been made in our understanding of the replication of cell culture-adapted HAV, prior to the making of the present invention, reproducible growth of wild-type human HAV in cell culture was not reported. Cell culture-adapted HAV readily infects cells and replicates within one to two weeks. Wild-type HAV infects and replicates in only a few cells out of large cell populations, does not spread to other cells in the culture, and remains latent for a long period during which it accumulates cell culture adapting mutations that allow the resulting virus, which is not longer a wild type strain, to spread through the cell culture. There was no accessible system to select those cells that support growth of wild-type HAV.

An HAV genome tagged with a selectable marker gene allows cells that replicate such a tagged HAV genome to express the marker gene, and thus virus replicating cells can be selected from a large background of population of cells that do not support viral replication. Thus, the present invention allows adequate growth of HAV virus sufficient to allow rational development of an attenuated vaccine strain. Once a good HAV candidate vaccine strain is developed, the method of the present invention may be applied to that strain to select a suitable cell line for growing that vaccine strain for vaccine production.

The HAV genome is able to accommodate added nucleotides or genes. The primary polyprotein cleavage site at the 2A/2B junction will tolerate insertion of exogenous nucleotides; we have demonstrated that cell culture-adapted variant HAV was able to tolerate with insertion of an exogenous sequence of sixty nucleotides in that junction and was stable for at least six serial passages (1, 41). A recombinant HAV containing a bleomycin resistance gene inserted at the 2A/2B junction was stable in cell culture without selection for at least five passages, the limit of the experiment (1).

The rescue of wt HAV from cells transfected with infectious in vitro synthesized full-length RNA transcripts is highly inefficient (11). Rescue of wt HAV from direct RNA transfection of marmoset livers (14) is possible, but this procedure is cumbersome and expensive. In addition, growth of the rescued wt HAV in cell culture is problematic because the virus is unstable and accumulates cell culture-adapting mutations that result in its attenuation (11, 12, 13, 15, 19). Therefore, experimentation with wt HAV is extremely difficult, and this hampers further advances in understanding of the pathogenesis of HAV. To circumvent this problem, we explored some alternatives to enhance the marginal infectivity of wt HAV cDNA in cell culture.

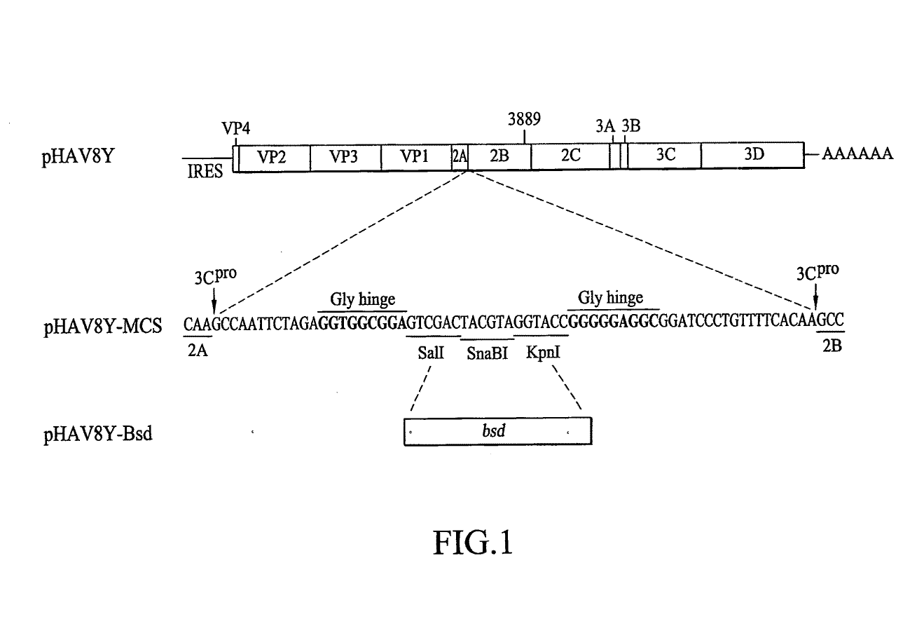

In the present invention a recombinant wt HAV coding for a selectable marker could be used to select cells expressing host factors required for its efficient and stable growth in cell culture. Similarly, an attenuated but not cell culture adapted HAV could be used to select cell lines that allow its efficient growth for vaccine purposes. A selectable marker is inserted into the wt HAV genome in-frame with the polyprotein. First, A polylinker coding for the unique SalI, SnaBI, and KpnI sites flanked by Gly hinges and 3 C^{pro} protease sites at the 2A/2B junction of the HAV cDNA in pHAV8Y is introduced; this construct is called pHAV8Y-MCS (FIG. 1). The HAV8Y background is used because this virus contains the cell culture adapting 2B-A216V mutations that enhances growth in cell culture (15) but does not affect the virulence of HAV (14). The blasticidin-resistance gene bsd lacking translation initiation and termination codons was inserted into the SalI and KpnI sites of pHAV8Y-MCS (FIG. 1). The resulting construct, termed pHAV8Y-Bsd, contained the bsd gene inserted in-frame with the HAV polyprotein. Therefore, processing of the polyprotein by the virus encoded 3C^{pro} is considered to result in the release of the bsd encoded deaminase (Bsd).

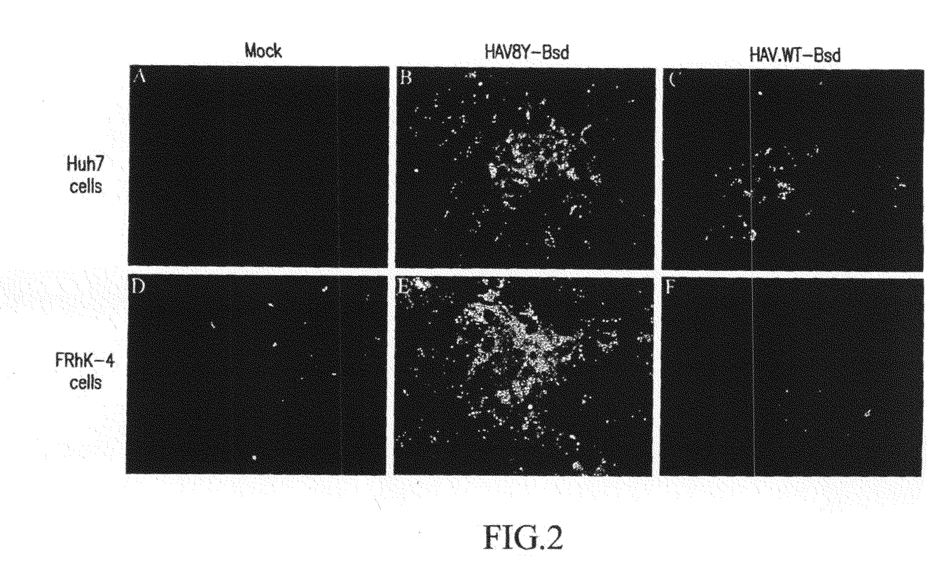

In general, wild-type (wt) HAV does not grow in cell culture but, when it does, it tends to accumulate cell culture-adapting mutations that result in its attenuation. For instance, the prototype wt HM-175 strain of HAV required months to grow in African green monkey kidney primary cultures (10) and accumulated 23 mutations that attenuated the virus in marmosets and chimpanzees. On the other hand, the present invention provides cells, exemplified by Huh7 cells and cell lines derived therefrom, that are permissive for wt HAV growth. When cells permissive for growth of wild-type HAV are infected with wt HAV or transfected with wild-type HAV genomic nucleic acids, HAV antigens can be detected by IF analysis in few days after infection (FIG. 2).

The time at which HAV antigens are detectable depends on the multiplicity of infection (MOI). At a high MOI, or example at about 10 or above, HAV antigens may be detected in one day. Decreasing the MOI delays the appearance of detectable HAV antigens; as long as one week in culture may be required. At a MOI of 0.1 to 10, HAV antigens can typically be detected by IF within 1 week. In a typical embodiment of cells of the invention, more than 20% of cells will express human HAV antigens, when assessed by immunofluorescence, within one week when the culture is begun with a multiplicity of infection of from 0.1 to 1.

The examples herein also demonstrate that Huh7-A-I cells, a selected subline of Huh7 cells, are highly susceptible to wt HAV growth (FIG. 5A). Interestingly, wt HAV grew 10-fold better in Huh7-A-I cells than in parental Huh7 cells. Wild-type HAV was stable in Huh7 and Huh7-A-I cells and does not accumulate cell culture-adapting mutations. These findings are confirmed by lack of growth of the wt HAV in FRhK-4 cells (FIG. 5A) and nucleotide sequence analysis of virus recovered from the cultured Huh7 and Huh7-A-I cells (FIG. 5B). The efficient and stable growth of wt HAV in Huh7-A-I cells clearly indicates these cells do not exert the strong selective pressure found in most other cell lines for the accumulation of cell culture-adapting mutations. Indeed, Huh7-A-I cells most likely contain cellular factors similar to those found in human liver cells that allow the growth of wt HAV.

Rescue of wt HAV from full-length cDNA has previously been difficult because in vitro transcripts are only marginally infectious in cell culture in cell lines utilized in the prior art. FRhK-4 cells transfected with in vitro RNA transcripts derived from the wt HM-175 full-length cDNA required 132 days to show HAV-specific IF in only 5% of the cells (15). The inefficient growth of wt HAV most likely forces the selection of mutants that replicate more efficiently in cell culture. Transcripts of a wt HAV construct containing the critical cell culture-adapting mutation 2B-A216V were very slightly more infectious in transfected FRhK-4 cells, resulting in the infection of a few single cells; the infection did not spread to the whole culture (11). To rescue wt HAV, Emerson et al. directly inoculated marmoset livers with a mixture containing cDNA and full-length genomic RNA transcripts of wt HAV containing the 2B-A216V mutation (14). FRhK-4 cells inoculated with fecal suspensions from the liver-transfected marmosets became infected in a short time (14), indicating that the 2B-A216V mutation enhanced the infectivity of the wt HAV in cell culture. The results of the examples are consistent with these findings in that HAV8Y-Bsd could not be rescued from transfected FRhK-4 cells but we were able to infect these cells with stocks of HAV8Y-Bsd derived from Huh7 cells (FIG. 2). Taking these data together, it is clear that the in vitro transcripts are less efficient than viral particles in establishing an infection in FRhK-4 cells. Although effective in rescuing wt HAV, the direct transfection of marmoset livers is a complicated and expensive technique, which contrasts with the simplicity of deriving wt HAV from transfected Huh7 or Huh7-A-I cells as exemplified herein.

Some considerations related to controlling experiments utilizing transfection of mammalian cells in culture using transcripts of HAV are described in Emerson et al. (1993) (ref. 13), see esp. pp. 478-479.

Molecular clones of wt and attenuated HAV have been available for approximately two decades (6-8). However, the lack of a robust cell culture system that could allow the rescue and efficient growth of wt viruses limited the use of reverse genetics to understand the pathogenesis of HAV and develop cost-effective attenuated vaccines. The availability of the highly permissive Huh7-A-I cells for wt HAV growth allows the application of reverse genetics to the study of the pathogenesis of HAV and development of an effective live, attenuated vaccine for HAV.

Most cell culture-adapted HAV strains do not cause cytopathic effects (CPE), but it has been shown that some strains can cause CPE in cell lines in which HAV replicates fast (28, 32, 40) triggering apoptosis (4, 17, 26). In the examples herein, wt HAV establishes persistent infections in Huh7 (FIG. 2) and Huh7-A-I (data not shown) cells without causing CPE. Construction of a wt HAV containing the blasticidin selection marker allows screening for cell lines that could support virus replication, which resulted in the identification of the human hepatoma Huh7 cell line as permissive for wt HAV growth. A similar construct containing a gene for resistance to bleomycin, a DNA intercalator, was reported previously (1) but was not suitable for selection of permissive cells because of the slow nature of the bleomycin selection. Indeed, when we transfected Huh7 cells and cells of other lines with an HAV construct containing the bleomycin selection marker instead of the blasticidin marker and treated cells 24 h after transfection with the corresponding antibiotic, we could not select surviving antibiotic-resistant cells (Konduru and Kaplan, manuscript in preparation). Therefore, the use of bsd as a rapid and effective selectable marker allowed the HAV constructs to confer antibiotic resistance to the transfected cells that supported at least a minimal level of viral replication, which is not the case for other selectable markers such as the bleomycin resistance gene.

In general, a selectable marker gene for use in the present invention is one that allows for selection of transfected cells within one week. This is in contrast to a “slow” selection, such as zeocin or neomycin resistances, which typically take two weeks to one month.

Resistances useful in the present invention include resistance to translational inhibitors, such as puromycin and its derivatives, and of course, blasticidin as shown in the Examples. Bcl-2 genes provide resistance to several known cancer treatments, and a vector expressing a Bcl-2 gene would provide resistance to drugs that induce apoptosis.

The HAV constructs coding for bsd are an excellent genetic tool that will allow identification of genes required for the growth of HAV, development of rapid titration and neutralization tests for research and diagnosis, and of host cells that support more rapid growth and/or growth to higher titers of vaccine strains of HAV. In general, by increasing the blasticidin (or other selective agent) concentration, it is possible to select cells that support higher levels of HAV replication. That is, a cell line that is first selected using the recombinant virus of the invention and a certain level of blasticidin may then be cultured using a higher level of blasticidin in the medium. Alternatively, a virus stock may be made from the first round selected cells, then this virus stock used to infect a second culture of naïve cells, which are then selected for blasticidin resistance at a higher concentration of blasticidin. For instance, for the first round of transfection and selection, 1 μg/ml of blasticidin might be used, followed by successive culture at one or more concentrations as 2, 5, 10, 20 and up to 50 μg/ml of blasticidin. Of course, the steps that are used may be varied as appropriate to the particular virus and cells being used. Cells surviving at the higher level of blasticidin (or other selection) may express a phenotype of supporting more rapid growth of human HAV or of producing a higher end titer of HAV. Similarly, virus obtained from cells cultured at higher concentrations of blasticidin may contain mutations that allow for more rapid replication in culture and/or growth to higher titer.

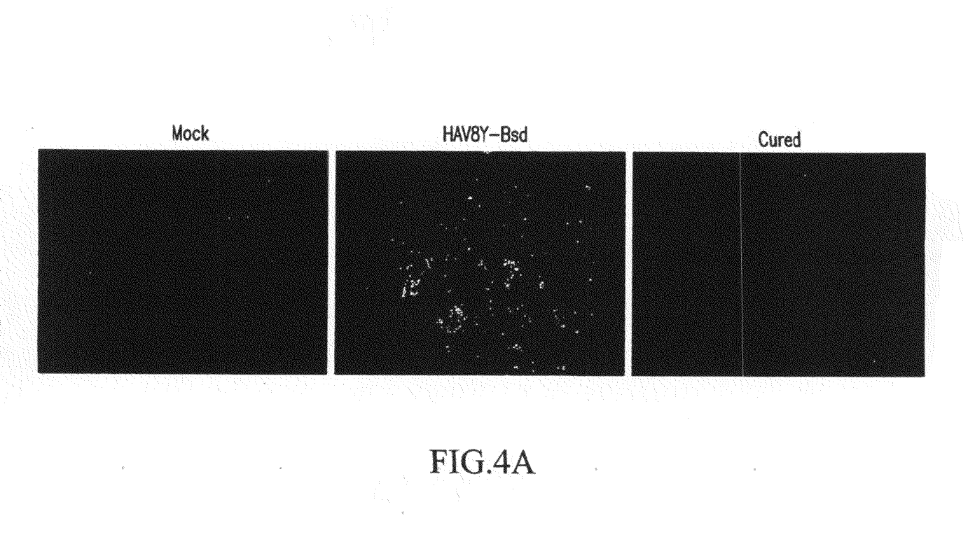

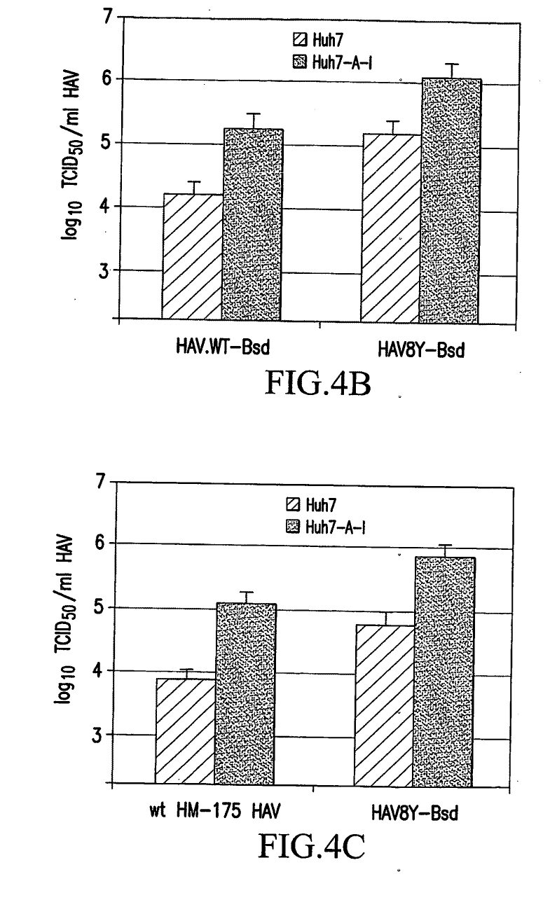

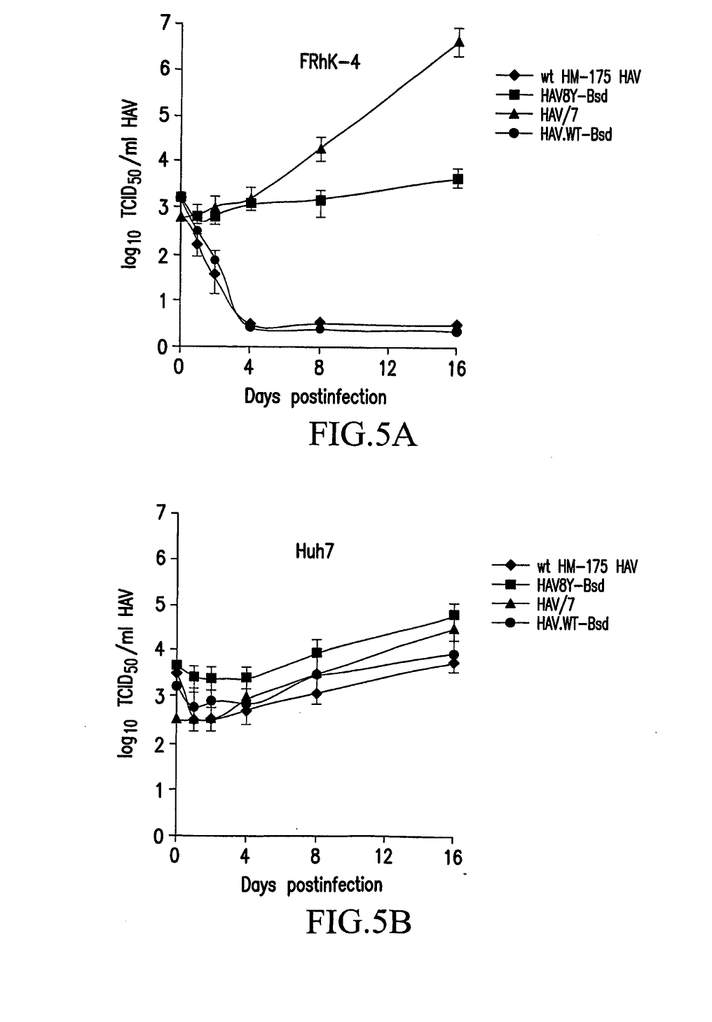

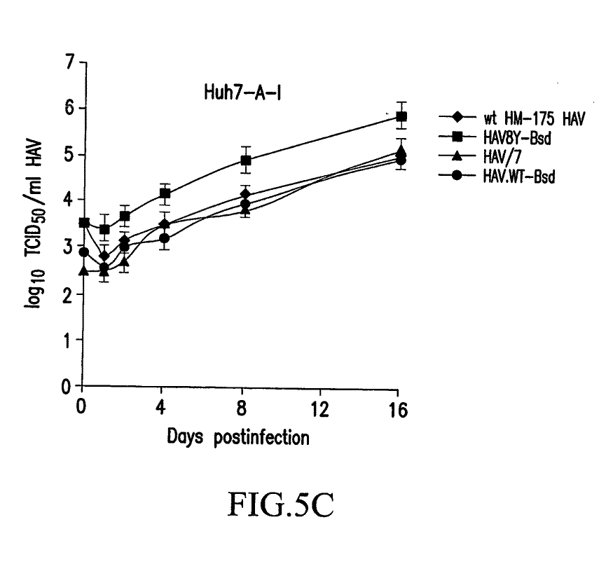

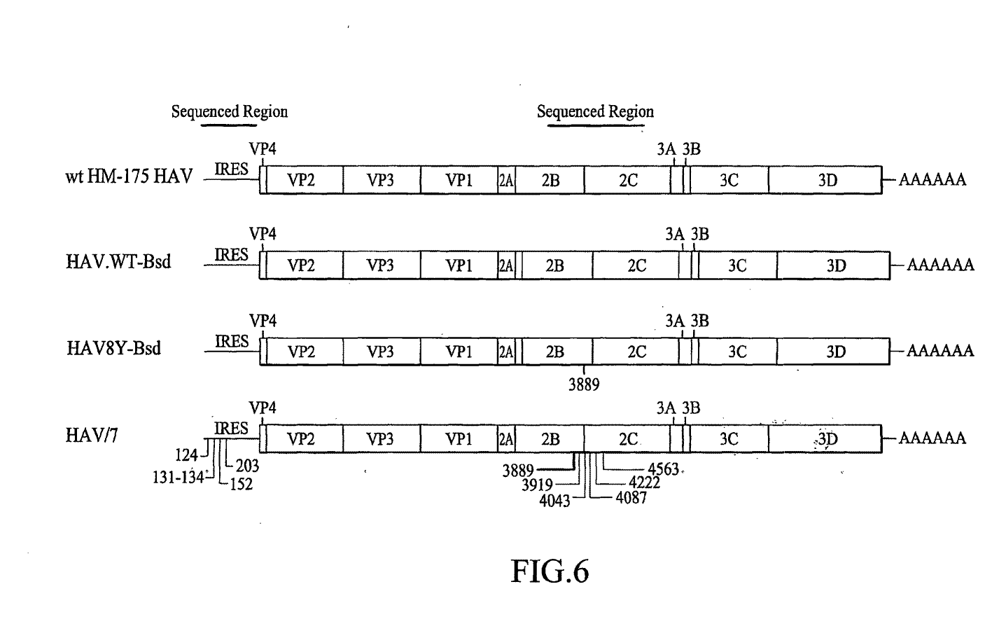

The interferon-cured Huh7-A-I cells exemplified herein are more susceptible to wt HAV infection than the parental Huh7 cells (FIG. 4, B and C), and supported higher levels of wt HAV growth (FIG. 5). Huh7 cells have been used to efficiently grow Hepatitis C Virus (HCV) recombinants containing the neomycin (Neo) resistance gene (29). It has been demonstrated that after IFN curing, the transfected Huh7 cell clones were able to support elevated levels of HCV replication (51).

Since both wt HAV and HCV are hepatotropic viruses and replicate efficiently in sublines of Huh7 cells, it is likely that this human hepatoma derived cell line expresses hepatocyte-specific cellular factors required for the in vivo growth of these viruses. However, the Huh7 cell sublines selected using the wild-type HAV recombinant viruses comprising the blasticidin selectable marker had similar susceptibility to HCV replicons as the parental Huh7 cells. This indicates that the Huh7 cell sublines selected using the HAV vectors are different from the Huh7 sublines selected using the HCV replicons. HAV vectors encoding functional and effective selectable markers and the identification of cell lines capable of supporting the efficient growth of virulent and pathogenic wt HAV provide tools for the study of HAV replication and pathogenesis and also allow the development of cost-effective attenuated live virus vaccines.

One aspect of the invention is represented by a recombinant Hepatitis A Virus nucleic acid comprising the nucleotide sequence of a wild-type HAV genome (SEQ ID NO: 1) or the nucleotide sequence of a HAV genome in which a codon encodes valine at amino acid 216 of the 2B protein. In the instance of the HAV genome encoding valine at amino acid 216 of the 2B protein, the sequence is that of HAV8Y, which bears a mutation at residue 3889 changing a cytosine residue to a thymine residue (52). The present invention also contemplates recombinant HAV genomes having mutations at positions 4087 and/or 4222 as a complement to or substitution for the mutation at nucleotide 3889 (13).

The recombinant HAV nucleic acid of this embodiment of the invention further comprises a “cloning site” or “multiple cloning site” that is a nucleotide sequence representing at least one unique restriction enzyme site located between nucleotides encoding protease 3C^{pro} cleavage sites that is in turn located at the junction of the 2A and 2B genes of the recombinant Hepatitis A Virus. In some embodiments, the cloning site also be flanked by nucleotides encoding “glycine hinge” amino acids. A glycine hinge is formed by a short sequence, from 3 to 5 amino acids, of small hydrophobic amino acids, such as glycine and/or alanine. The hinge may include a serine amino acid. Thus, for example, a “glycine hinge” may be a sequence of -gly-gly-gly- or -gly-ala-gly-, or -gly-ser-gly- or -ala-gly-gly or any other combination thereof.

The recombinant HAV nucleic acid of the invention may contain a heterologous nucleic acid in a position located between nucleotides encoding protease 3C^{pro} cleavage sites that is in turn located at the junction of the 2A and 2B genes of the recombinant Hepatitis A Virus. In such an embodiment it is also preferred that the cloning site also be flanked by nucleotides encoding “glycine hinge” amino acids. The heterologous nucleic acid sequence may be one that is inserted into the cloning site using the normal methods of restriction enzyme digestion and ligation of desired nucleic acid fragments. Alternatively, the cloning site may be absent and the heterologous nucleic acid may be inserted by methods known in the art such as overlap PCR.

The heterologous nucleic acid that is inserted is not particularly limited, and it can be one that encodes any desired amino acid sequence or functional RNA. Heterologous sequences representing more than one expression product may be inserted.

In a preferred embodiment, which may find use for rescue of cells that are very permissive for growth of wild-type HAV, the heterologous nucleic acid encodes a protein conferring a selectable or screenable phenotype upon a cell that expresses said protein. Such a selectable or screenable phenotype may be conferred by a fluorescent protein or a protein producing a colored reaction product, or more preferably, the phenotype is one of resistance to an antibiotic. In such a case, the antibiotic is preferably one that interferes with protein translation in a mammalian cell, such as blasticidin, puromycin or a puromycin derivative. Another preferred selection is a compound that induces apoptosis, and a corresponding resistance gene, such as BCL-2. The antibiotic resistance or other selectable marker allows for selection of cells that have taken up the recombinant HAV genome and are able to replicate it so as to allow proliferation of the recombinant HAV genome.

A further embodiment of the invention is a DNA expression vector comprising a DNA recombinant Hepatitis A Virus nucleic acid as described above that is operatively linked to a promoter for transcription of a genomic RNA other recombinant Hepatitis A Virus. In this embodiment of the invention, the promoter is preferably one that is suitable for in vitro transcription of the viral genomic RNA. Such promoters are well-known in the art, for example the SP6 promoter or the T7 promoter, each of which can be utilized in vitro together with their respective purified RNA polymerases.

The form of the recombinant HAV nucleic acid is not particularly limited. That is, it may be present as an isolated nucleic acid, as nucleic acids that are in the form of a mixture of cultured cells and their products, or as part of an assembled viral particle. In instances where the recombinant HAV nucleic acid encodes a heterologous protein that is a vaccine antigen, either derived from a hepatitis or other virus, or from any other organism, the viral particle comprising the recombinant HAV genome may find use as a vaccine.

The recombinant HAV genome of the invention is useful in vaccine development. The recombinant HAV genome may be further modified by introduction of single mutations or combinations of mutations to study the effect of such mutations on the replication rate and production of infectious virus. The cells selected as permissive for growth of HAV may be used to support the growth of wild-type HAV having candidate attenuating mutations introduced, thereby enabling the growth of sufficient virus for pre-clinical testing of in vivo attenuation and immunogenicity of candidate live vaccine strains in animal models. The cell lines of the present invention confer the advantage over cells previously used to grow HAV that they do not select for virus having culture-adapting mutations and therefore candidate vaccine strains can be grown in them without the complication of accumulation of additional mutations. Alternatively, the permissive cell lines identified by the present invention may be studied to identify host cell determinants of HAV virulence and pathogenicity, for example by comparing the protein expression profiles of such permissive cells with a non-permissive cell line such as FRhK-4 cells. Materials and methods for performing such expression profiling, for example, 2-D protein electrophoresis, are considered well-known in the art.

The present invention may also be used to molecularly clone and identify cellular factors that allow the growth of wild-type HAV, or that allow growth of vaccine strains of HAV, in cells. One approach that may be used to accomplish these aims is to prepare a library from a hepatoma cell line, for instance from Huh7 cells, or from any other cell found to be permissive for growth of wild-type HAV. Such cells are, for example, primary liver tissue cultured cells or cells of liver tissue per se, or monocytes or mucosal epithelial cells. The library may also be made from cells used to grow a vaccine strain, such as MRC-5 cells or Vero cells. The library should be made in a vector that allows for expression of the inserted nucleic acid in mammalian host cells transfected with the library. Several such vectors are known in the art. Examples of episomal vectors are EBV-P1 based vectors such as pDR2 (Clontech) or the pEAK8 or pEAK12 vectors. Episomal vectors provide the advantage that the inserted DNA is readily isolated from preparations of plasmid DNA.

Integrating vectors are also known. If an integrating vector is used, then the inserted nucleic acids may be recovered by, for example, polymerase chain reaction using the primers derived from vector arm sequences.

The library is then transfected into a cell line that is non-permissive for replication of wild-type HAV and transformed cells are selected for the presence of the library DNA, e.g. by a marker gene present in the library vector. The library is then transfected with recombinant HAV having a wild-type background and having an inserted selectable or screenable marker gene, in the fashion described herein. See, e.g. the construct illustrated in FIG. 1. Selection or screening for the marker gene carried by the recombinant HAV identifies members of the library that express genes encoding cellular factors that support the growth of wild-type HAV.

This method may be modified, for example, by transforming the non-permissive cells with the library at the same time as infecting them with viral particles comprising the recombinant HAV of the invention and then selecting for cells rendered permissive using both selection markers at the same time.

The present invention also provides a method for selecting a cell permissive for growth replication of Hepatitis A Virus, and especially one that is permissive for growth of wild-type HAV. Such a method comprises transfecting cultured cells with the recombinant Hepatitis A Virus nucleic acid of the invention and then selecting or screening the transfected cells for the phenotype conferred by the recombinant Hepatitis A Virus. A cell exhibiting the selected or screened phenotype is deemed to be permissive for growth and replication of Hepatitis A Virus.

The selected cells can be further cultured to provide stocks for storing of the cell line and for cloning of cells to provide pure cell lines. Cloning of cells for establishment of single-clone cell lines is considered well-known in the art. The further culture of the cells for purposes of establishing initial stocks of permissive cells may be conducted in the presence of the reagent for which selection is performed initially, so as to maintain the presence of the recombinant HAV genome in the initial cell line.

Alternatively, the further culture of the cells may be performed in the absence of the selection reagent and also optionally in the presence of an interferon so as to promote curing of the genome of the cell line of the recombinant HAV genome. Such a “cured” cell line provides a useful host for culture of attenuated strains of hepatitis virus, especially HAV, for production of vaccines and for testing of samples obtained from the environment (including food samples) and from patients, and for determination of the content of such samples of replicating HAV by culturing methods.

In establishing a cell line that is permissive for growth and replication of a wild-type HAV, it is desirable to further test the ability of the cured cell line to again support growth of either wild-type HAV or of a mutated HAV. Such a mutated HAV may be the HAV8Y strain that contains only the A261V mutation in the 2B protein that is considered the minimal mutation required for adaptation of the HAV to cell culture. The mutated HAV may be one that includes any other mutations that confer the ability to grow and replicate well in cultured cells. Alternatively, the mutated HAV may be an attenuated strain that is useful as a vaccine for human use.

In all instances of the invention, the HAV nucleic acid, whatever its form, may be introduced into cells either by infection with viral particles comprising the desired HAV nucleic acid, or by transfection of cells with HAV nucleic acid in purified or partially purified form using methods well-known in the art, such as by lipofection or electroporation. HAV nucleic acids may be introduced into cells in either RNA form or DNA form, depending upon the nature of a vector used. RNA forms of HAV genomic nucleic acids may be generated by in vitro transcription as described above. Alternatively, if the HAV genomic sequence is operatively linked to a promoter effective in mammalian cells within a DNA vector, such may be used to transfect mammalian cells which in turn will transcribe the HAV genomic nucleic acid in culture or in vivo.

The present invention also provides a method for selecting a cell permissive for growth and replication of Hepatitis A Virus. Such a method comprises transfecting cultured cells with the recombinant Hepatitis A Virus nucleic acid of the invention and then selecting or screening the transfected cells for the phenotype conferred by the recombinant Hepatitis A Virus. A cell exhibiting the selected or screened phenotype is deemed to be permissive for growth and replication of Hepatitis A Virus.

The ability of a cell to be infected by a wild type or attenuated Hepatitis Virus, especially Hepatitis A Virus may be assessed by contacting the cell with viral particles comprising the recombinant HAV nucleic acids of the invention and determining if the virus is replicating within the cell. Such determination can be made, for example, by IF assay for Hepatitis Virus (especially HAV) antigens in cultures of the contacted cells, or by measuring the titer of virus that accumulates in the culture over time.

The selected cells may be further cultured under conditions that provide for curing the selected cell of the Hepatitis A Virus nucleic acid.

The selected cells, before or after curing, may be tested for their ability to be infected by and to replicate wild-type HAV and/or HAV having attenuating mutations or cell-culture adapting mutations.

The present invention also encompasses cells and cell lines that are permissive for growth of wild-type HAV. Such cells may be obtained by the above-described method. A preferred cell line of the invention is one that is derived from a human hepatoma cell or from a normal human liver cell.

A preferred embodiment of the invention is a mammalian cell line comprising Huh7 cells that have been transfected with a recombinant Hepatitis A Virus nucleic acid comprising a nucleotide sequence encoding a protein conferring a selectable or screenable phenotype upon a cell that expresses said protein. The selectable marker sequence is located between nucleotides encoding protease 3C^{pro} cleavage sites that is in turn located at the junction of the 2A and 2B genes of the recombinant Hepatitis A Virus. The transfected cells are selected for the marker sequence and then subsequently cured of the recombinant Hepatitis A Virus. The resulting cells are permissive for replication of Hepatitis A Virus.

A human hepatoma cell line Huh7-A-I of the invention has been deposited at the American Type Culture Collection, P.O. Box 1549, Manassas, Va. 20108, USA under the terms and conditions of the Budapest treaty on Jun. 7, 2005 under the accession number PTA-6773.

The present invention also includes methods for producing a Hepatitis A Virus comprising infecting a cell with Hepatitis A Virus particles, or transfecting a cell with a nucleic acid representing the genome of a Hepatitis A Virus, culturing the infected or transfected cell to provide for replication of the Hepatitis A Virus, and separating particles of the Hepatitis A Virus from the cultured cells. The cell utilized is one that is that is permissive for growth and replication of wild-type HAV and/or a cell that provides for titers of an attenuated HAV strain of 10⁷ TCID₅₀/ml, preferably 10^{7.5}, 10⁸, or higher.

The present invention also provides methods for assaying a sample for infectious Hepatitis A Virus. Such method comprise contacting the sample with cells from a cell line of any one of the invention, culturing the cells, and then determining the presence of Hepatitis A Virus in the sample. The presence of HAV in the sample may be detected by any methods known in the art, such as titering the virus present in the cultured cells by contacting a sample of a supernatant of the culture with mammalian cells that may be infected by Hepatitis A Virus and counting infected cells. Alternatively, HAV nucleic acids may be detected and/or quantitated in the cultured cells or in supernatants from the cultured cells by performing a polymerase chain reaction using primers specific for Hepatitis A Virus nucleic acids and a nucleic acid sample prepared from cells or the supernatant of the culture as a template. Both qualitative and quantitative PCR methods are considered to be known in the art. Proteins specific for HAV may be detected and/or quantitated by assaying for the presence of at least one protein specific to Hepatitis A Virus by an immunoassay method. Monoclonal antibodies specific for HAV and a number of immunoassay methods, both qualitative and quantitative, are considered known in the art.

### EXAMPLES

The following examples serve to illustrate the invention and are not limiting thereof. The invention is limited only by the claims following.

### General Materials and Methods Used in the Examples

Cells and viruses. Human hepatoma Huh7 cells with various passage history, obtained from Drs. D. Taylor and C.Hsia, FDA, Bethesda, Md., were grown in Dulbecco's modified Eagle's medium (DMEM) supplemented with 10% fetal bovine serum (FBS). The continuous clone GL37 of African green monkey kidney cells (AGMK) (39) was grown in Eagle's minimal essential medium (EMEM) supplemented with 10% FBS. Human HeLa cells and AGMK Vero cells, obtained from ATCC, were grown in DMEM with 10% FBS. Rhesus monkey FRhK4 cells, obtained from Dr. S. Emerson, NIH, Bethesda, Md., were grown in DMEM supplemented with 10% equine serum. Chinese hamster ovary (CHO) cells deficient in the enzyme dihydrofolate reductase (dhfr), obtained from ATCC, were grown in Iscove's medium containing 10% FBS and supplemented with 100 μM hypoxanthine and 16 μM thymidine (Sigma Chemical Co.). Mouse liver cell line MMH-D3 (53) derived from transgenic mice carrying a truncated cytoplasmic form of the human Met gene, were grown in RPMI 1640 medium with 10% FBS, 10 μg/ml insulin, 50 ng/ml EGF, and 30 ng/ml IGF2 growth factors on collagen coated flasks. Human Jurkat cells, obtained from ATCC, were grown in RPMI 1640 medium with 10% FBS. All cell lines were grown in a 5% CO₂ incubator at 37° C.

Stool derived wild-type (wt) HM-175 strain of HAV was obtained from Dr. S. Feinstone, FDA, Bethesda, Md. A wt HM-175 strain of HAV containing a Ala to Val change at position 216 of protein 2B (2B-A216V) (11, 14, 18), termed HAV-8Y, was derived from Huh7 cells transfected with in vitro run-off SP6 polymerase transcripts from pHAV.

Plasmids and constructs. The infectious cDNA of HAV-8Y, which codes for wt HM-175 HAV containing the A216V change in the 2B protein, in pHAV8Y (11, 14) and the infectious cDNA of the cell culture-adapted HM-175 strain of HAV in pHAV/7 (5) are under the control of the SP6 RNA polymerase.

Plasmids were constructed using PCR and standard molecular biology methods (35). PCR DNA fragments were amplified using Pfu Turbo Hotstart polymerase (Stratagene) as recommended by the manufacturer. Amplifications were done in 25 cycles of 95° C. for 30 sec, 50° C. for 1 min, and 72° C. for 2 min. For overlap PCR, fragments were denatured at 94° C. and annealed at 45° C. in 1×PCR buffer for 2 min. Escherichia coli strain DH5α was transformed with the constructs, and plasmids were purified by chromatography (Quiagen) as suggested by the manufacturer. Constructs were verified by automatic nucleotide sequence analysis using the ABI Prism BigDye terminator cycle sequencing ready reaction kit (Applied Biosystems) and the ABI Prism (model 3100) analyzer (Applied Biosystems). The following plasmids were constructed:

pHAV8Y-MCS. A polylinker containing unique restriction sites SalI, SnaBI, and KpnI flanked by three-residue Gly hinges designed to facilitate processing of the two adjacent 3C^{pro} cleavage sites and cleavage sites for the HAV protease 3C^{pro} (32, 38) was introduced into the 2A/2B junction of the HAV infectious cDNA in pHAV8Y using overlap PCR (FIG. 1). Forward PCR primer A (5′-GTTTTATTTTCCCAGAGCTCCATTGAACTCAA-3′) (SEQ ID NO: 2) corresponding to nts 2975-3006 of HAV coding for the C terminus of protein VP1 and containing a naturally occurring SacI restriction site, and reverse PCR primer B (5′-GGTACCTACGTAGTCGACTCCGCCACCTCTAGAATTGGCTTGTGAAAACAGTCCC TTCTTCATTTTCCTAGG-3′) (SEQ ID NO: 3) coding for nucleotides (nts) 3213-3242 of HAV corresponding to the C terminus of the 2A protein, a synthetic 3C^{pro} cleavage site, and the polylinker described above plus a three-residue Gly hinge, were used to amplify fragment I using pHAV8Y as template. An additional PCR fragment II was amplified using the same HAV cDNA as template and oligonucleotides C (5′-GACTACGTAGGTACCGGGGGAGGCGGATCC CTGTTTTCACAAGCCAATATTTCTCTTTTTTATACTGAGGAG-3′) (SEQ ID NO: 4) and D (5′-ATTTTTCCACATCTTGGATTTGCAAAATGCAAAATT-3′) (SEQ ID NO: 5) as PCR primers. The 5′ end 15 nucleotides of forward PCR primer C are complementary to the polylinker of oligonucleotide B followed by three codons of the Gly-hinge, a 3C^{pro} cleavage site, and nts 3243-3272 of HAV. Reverse PCR primer D codes for nts 4183-4217 of HAV and contains a naturally occurring PflMI restriction site. PCR fragments I and II were annealed and used as a template for the amplification of a larger fragment using the forward A and reverse D PCR primers. The resulting PCR fragment was gel-purified, digested with SacI and PflMI enzymes, and cloned into pHAV8Y cut with the same enzymes. The resulting construct was termed pHAV8Y-MCS.

pHAV8Y-Bsd. The blasticidin resistance gene Bsd was cloned into the polylinker of pHAV8Y-MCS. A DNA fragment was amplified from pTracer-CMV/Bsd (Invitrogen) using synthetic oligonucleotide primers 5′-GTCGACGTCGACCAGGCCA AGCCTTTGTCTCAAGAA-3′ (SEQ ID NO: 6) and 5′-CGGTTAGGTACCGCCCTCCCACACATAA CCAGAGGG-3′ (SEQ ID NO: 7), which introduced SalI and KpnI restriction sites at the 5′ and 3′ ends of the gene, respectively, and eliminated the translation initiation and termination codons of bsd. The resulting PCR fragment was gel-purified, digested with SalI and KpnI, and cloned into pHAV8Y-MCS digested with the same restriction enzymes. The resulting construct was termed pHAV8Y-Bsd, and encodes the Bsd resistance protein inserted between the 2A and 2B genes in-frame with the rest of the HAV polyprotein.

pHAV.WT-Bsd. The 2B/A216V residue in HAV8Y was back-mutated to the Ala residue found in natural isolates of wt HAV. Overlap PCR was performed using forward primer A1 (5′-GAGTCATGAATTATGCAGATA-3′) (SEQ ID NO: 8) coding for nts 3874-3894 of wt HAV and reverse primer A2 (5′-AACCAATATCTGCATAATTCA-3′) (SEQ ID NO: 9) coding for nts 3900-3880 of wt HAV, both coding for an Ala codon (underlined) at position 216 of 2B. Two overlapping PCR cDNA fragments were amplified from pHAV8Y-Bsd using PCR primers A and A2, and PCR primers A1 and D, respectively. These two PCR cDNA fragments were denatured, annealed, and used as templates for the amplification of a longer PCR fragment using primers A and D that was digested with SalI and PflMI, gel-purified, and cloned into the SalI and PflMI sites of pHAV8Y-Bsd. The resulting construct was termed pHAV.WT-Bsd.

Immunofluorescence analysis. Mock- and HAV-infected cells grown in 8-well chamber slides at 35° C. were fixed with cold acetone for 30 min, air dried, blocked with 2% FBS in PBS, and stained with murine anti-HAV neutralizing monoclonal antibodies (Mabs) K2-4F2 and K3-4C8 (30) and FITC-conjugated goat anti-mouse antibody (KPL Inc). Fluorescent micrographs were taken with a Zeiss Axioscope microscope at a magnification of ×400 with an oil immersion objective.

RNA transfections and HAV infections. Full-length HAV RNA transcripts were synthesized in vitro using SP6 RNA polymerase (Amersham Pharmacia) and plasmid templates linearized at the HaeI site downstream the poly(A) of the HAV cDNA (7, 41). Yield (about 5-10 μg) and quality of in vitro synthesized RNA transcripts were examined by electrophoresis in a 1% agarose gel. Subconfluent cell monolayers grown in 25 cm² flasks were transfected with the RNA transcripts using DEAE-dextran as a facilitator (33). After 30 min at room temperature, monolayers were washed, fresh medium was added, and cells were incubated at 35° C. One day after transfection, cells were split 1:2 and grown in selection medium containing 2 μg/ml blasticidin (Invitrogen). Cells were split 1:5 weekly into 25 cm2 flasks and 8-well Permanox chamber slides (Nunc, Inc) for immunofluorescence (IF) analysis. Monolayers with 80% of the cells expressing HAV antigens as assessed by IF analysis were subjected to three freeze-and-thaw cycles, cell debris was pelleted by low-speed centrifugation, and supernatants containing the virus were stored at −70° C.

To infect cells, 50-80%-confluent cell monolayers were inoculated with a multiplicity of infection (MOI) of 1-2 TCID₅₀/cells in 25-cm² flasks. Infected cells were grown at 35° C. in a CO₂ incubator. At 24 h postinfection, blasticidin (2 μg/ml) was added to the medium to select for cells infected with HAV constructs including the bsd resistance gene. To prepare larger virus stocks, cells were trypsinized one week after infection and grown in 225-cm² flask for two more weeks. For one-step growth curve analysis, 6-well plates were infected using the same conditions described above, and plates were frozen at −70° C. at different time points. After the last time point was collected, plates were thawed and viral stocks were prepared as indicated above.

HAV titer determination. HAV titers were determined by an ELISA endpoint dilution assay in 96-well plates containing 20-50% confluent cell monolayers. Eight replicate wells were inoculated with 100 μl of 10-fold dilutions of HAV prepared in DMEM-10% FBS. The plates were incubated at 35° in a CO₂ incubator. Viral titers were determined by ELISA two weeks after infection. ELISA was performed by fixing cell monolayers with 90% methanol and staining with a 1:2,500 dilution of Mab K2-4F2 and a 1:25,000 dilution of peroxidase-labeled goat anti-mouse antibodies (KPL Inc.) TMB one-component substrate (KPL Inc.) (100 ml/well) was added, the plates were incubated at room temperature for 15 to 30 min, and the reaction was stopped with 1% H₂SO₄ (100 ml/well). Wells that developed at least 2 times the absorbance of the uninfected control wells were considered positive.

Alternatively, HAV8Y-Bsd and HAVwt-Bsd titers were determined by a blasticidin-resistance endpoint dilution assay in 96-well plates containing 20-50% confluent monolayers of Huh7 cells. Blasticidin (2 μg/ml) was added to the cell culture media of the 96-well plates 24 h after infection, and incubated at 35° C. under CO₂. Five to seven days after infection, the 96-well plates were inspected under the microscope and wells containing monolayers or live cells forming colonies were considered as positive. Viral titers of the ELISA and blasticidin-resistance endpoint titrations were determined using the Reed and Muench method (34).

Cure of HAV-infected cells with Interferon. Huh7 cells transfected with HAV8Y-Bsd synthetic transcripts and selected with 2 μg/ml blasticidin were treated with human leukocyte-derived interferon-αA/D (IFN-αA/D) (Sigma Chemical Co.) to eliminate the virus from the cells. Prior to IFN-αA/D treatment, cell were split twice in growth medium lacking blasticidin. Huh7 cells infected with HAV8Y-Bsd were grown in 12-well plates in the presence of 100, 250, or 500 U/ml of IFN-αA/D in the absence of blasticidin. Cells were split weekly in medium containing IFN-αA/D and, after the 3rd passage in the presence of IFN-αA/D, the presence of HAV antigens was assessed by IF analysis, the sensitivity of the cells to antibiotic treatment in 96-well plates containing cells grown in the presence of 0.5-10 μg/ml blasticidin, and production of infectious HAV by IF analysis of naïve Huh7 cells infected with cell extracts. Naïve and HAV8Y-Bsd-infected Huh7 cells were used as negative and positive controls, respectively, for the blasticidin-resistance test. Cells that did not immunofluoresce, were sensitive to Bsd treatment, and did not produce infectious HAV, were considered cured. These cells were named Huh7-A-I, and stored in liquid nitrogen.

Nucleotide sequence analysis. HAV RNA was extracted from viral stocks using Trizol (Invitrogen). HAV cDNA was synthesized using the Supercript-II kit (Invitrogen) as recommended by the manufacturer using HAV RNA as template, and HAV-specific synthetic primers coding for nts 4879 to 4900 or 580 to 600. The HAV cDNA fragments from the 5′-NTR were amplified by PCR using synthetic primers coding for nts 1 to 21 and 580 to 600. The HAV cDNA fragments from the 2B-2C region were amplified using synthetic primers coding for nts 3781 to 3880 and 4879 to 4900. PCR amplifications were done using the same conditions and polymerase used for the plasmid constructs. PCR DNA fragments were gel-purified and both cDNA strands were sequenced using the ABI Prism BigDye terminator cycle sequencing ready reaction kit (Applied Biosystems) and the PCR amplification primers described above plus an additional primers conding for nts 4185 to 4205 to sequence the 2B-2C region. Automatic sequencing was done in an ABI Prism (model 3100) analyzer (Applied Biosystems).

### Example 1

### Rescue of Wt HAV from Cells Transfected with In Vitro Transcripts

To rescue HAV8Y-Bsd, SP6 transcripts were transfected into Huh7, FRhK4, GL37, HeLa, Vero, CHO, MMH-D3, and Jurkat cells. One day after transfection, cells were split 1:6 and grown in media containing 1, 2, 4, or 5 ug/ml blasticidin. After 14 days of selection with 1 μg/ml blasticidin, a small number of blasticidin-resistant colonies grew in Huh7 cells transfected with HAV8Y-Bsd RNA but not in mock-transfected cells. Transfected Huh7 cells did not survive selection with higher concentrations of blasticidin (2, 4, or 5 ug/ml). All the other transfected cell lines did not survive treatment with blasticidin, which indicated that HAV8Y-Bsd could only be rescued from Huh7 transfected cells. IF analysis (FIG. 2) showed that the blasticidin-resistant Huh7 cells had the characteristic cytoplasmic granular fluorescence of HAV infected cells (B), which was not observed in mock-transfected cells (A). To assess the role of the 2B-A216V mutation in the growth of wt HAV in Huh7 cells, we back-mutated nucleotide 3889 of HAV8Y-Bsd from T to C to restore the sequence observed in natural isolates of the virus, naming the construct pHAV.WT-Bsd. Similar to the results with HAV8Y-Bsd, Huh7 cells transfected with RNA transcripts derived from pHAV.WT-Bsd treated with 1 μg/ml blasticidin for 14 days resulted in the selection of a small number of resistant colonies that contained HAV antigens (C). It was of interest to determine whether the viruses rescued from transfected Huh7 cells could grow in FRhK-4 cells. IF analysis showed that FRhK-4 cells were susceptible to infection with HAV8Y-Bsd (E) but not HAV.WT-Bsd (F), consistent with prior results that FRhK-4 cells do not support growth of wild-type virus, but are permissive for the culture adapted strain HAV-8Y (11, 12, 14). As expected, HAV antigens were not detected in mock-infected FRhK-4 cells (D). Our data indicate that wt HAV can be efficiently rescued from Huh7 cells transfected with in vitro synthesized transcripts derived from infectious cDNA, and that this rescue is independent of the important HAV cell culture-adapting mutation at position 3889 A216V in the 2B protein.

To analyze whether the IF positive cells produced infectious HAV that could transmit resistance to blasticidin, we prepared a viral stock from the Huh7 cells infected with HAV8Y-Bsd or HAV.WT-Bsd and infected naïve Huh7 cells. Approximately 50% of the Huh7 cells infected with HAV8Y-Bsd or HAV.WT-Bsd survived treatment with 1 μg/ml blasticidin for 5 days, which indicated that the functional bsd gene cloned into the HAV genome was packaged into infectious particles.

### Example 2

### Stable Growth of Wt HAV in Huh7 Cells

It was of interest to determine whether the strong pressure for selection of cell culture-adapting and attenuating mutations observed in most cell lines was also present in Huh7 cells. To study whether wt HAV containing the Bsd selectable marker could stably grow in Huh7 cells without accumulating cell culture-adapting mutations and maintaining the selectable marker, we performed nine serial passages of HAV8Y-Bsd and HAV.WT-Bsd in Huh7 cells in the presence of 1 μg/ml blasticidin. RT-PCR amplification and nucleotide sequence analysis of the HAV RNA extracted from the nine serial passages revealed that the inserted bsd gene was stable in both viruses. Nucleotide sequences of the 2B-2C and 5′-NTR regions of passage 9 HAV8Y-Bsd and HAV.WT-Bsd were identical to the parental cDNA showing that these viruses did not accumulate cell culture-adapting mutations in these 2 hotspots. To further assess the stability of wt HAV in Huh7 cells, we studied growth of wt HAV in FRhK-4 cells, which is dependent on the presence of the main cell culture-adapting mutation in nucleotide 3889. Passage 9 HAV8Y-Bsd and HAV.WT-Bsd were titrated in parallel in Huh7 and FRhK-4 cells using the blasticidin-resistance endpoint dilution assay in 96-well plates (FIG. 3). Similar titers of HAV8Y-Bsd were obtained in both cell lines whereas HAV.WT-Bsd titer in Huh7 cells was approximately 10⁴ TCID₅₀/ml but was undetectable in FRhK-4 cells. The lack of growth of HAV.WT-Bsd in FRhK-4 cells further confirmed that this virus did not accumulate cell culture-adapting mutations during the 9 serial passages in Huh7 cells. Huh7 cells supported the stable growth of wt HAV irrespective of the presence of the main cell culture-adapting mutations at nucleotide 3889.

### Example 3

### INFα-A/D Cured HAV8Y-Bsd-Infected Huh7 Cells are Susceptible to Wt HAV Infection

The blasticidin-resistant cells from infected with HAV.WT-Bsd in Example 1 were cured with interferon (9). To do so, blasticidin-resistant Huh7 cells infected with HAV8Y-Bsd were grown for several passages in the presence of 100, 250, or 500 IU/ml IFN-αA/D in medium lacking blasticidin. After seven passages, IF analysis showed that cells treated with 250 (FIG. 4A) or 500 U/ml (data not shown) of IFN-αA/D lost the HAV antigens whereas untreated control cells (FIG. 4A) and some cells grown in the presence of 100 IU/ml IFN-αA/D (data not shown) had the characteristic cytoplasmic fluorescence of HAV-infected cells. To determine whether the interferon-treated cells that lost the HAV antigens also became sensitive to blasticidin, the interferon-treated cells were grown in the presence of 0.5 to 10 μg/ml blasticidin for 10 days and the cultures were observed by microscope.

Control HAV8Y-Bsd-infected cells grew in the presence of blasticidin up to 8 μg/ml whereas the cells that lost the HAV antigens and control Huh7 cells died after treatment with 1 μg/ml blasticidin. In addition, HAV RNA was not detected by RT-PCR analysis in cells that lost the HAV antigens upon treatment with IFN-αA/D (data not shown). These data clearly indicated that HAV8Y-Bsd-infected Huh7 cells were cured from the HAV infection after treatment with INF-αA/D in the absence of blasticidin. To verify that there was no residual infectious HAV remaining in the cultures, the cured cells were passed twice in the absence of INF-αA/D and IF and sensitivity to blasticidin treatment analyses were performed, which showed that the cured cells did not have HAV antigens and were sensitive to blasticidin. The Huh7 cells cured from HAV8Y-Bsd-A infection with 250 U/ml IFN-αA/D were mamed Huh7-A-I and stored in liquid nitrogen.

To determine whether the Huh7-A-I cured cells were susceptible to wt HAV infection, HAV8Y-Bsd and HAV.WT-Bsd were titrated in Huh7-A-I and naïve cells using the blasticidin-resistance endpoint dilution assay (FIG. 4B). Both viruses produced 10-fold higher titers in Huh7-A-I than naïve Huh7 cells, which confirmed that the Huh7-A-I subline was more susceptible to wt HAV infection than the parental cell line. We also analyzed the susceptibility of both cell lines to infection with wt HM-175 strain of HAV isolated from human stools (wt HM-175 HAV). An ELISA endpoint dilution assay in 96-well plates showed that Huh7-A-I cells were also 10-fold more susceptible than parental Huh7 cells to wt HM-175 HAV infection (FIG. 4C). The increased susceptibility of Huh7-A-I cells to wt HAV infection was independent of the cell culture-adapting mutation at position 3889 as well as the presence of the Bsd selectable marker. Moreover, this is the first report of a cell line that is highly susceptible to infection with a natural isolate of wt HAV.

### Example 4

### The Cured Huh7-A-I Subline is Highly Permissive for Wt HAV Growth

A one-step growth curve analysis of wt HM-175 HAV, HAV8Y-Bsd, and HAV.WT-Bsd, and cell culture-adapted HAV/7 was performed in Huh7, Huh7-A-I, and control FRhK-4 cells. Time points were titrated by the ELISA endpoint dilution assay in 96-well plates containing Huh7-A-I cells (FIG. 5A). Cell culture-adapted HAV/7 grew efficiently to similar levels of approximately 10⁶-10⁷ TCID₅₀/ml in the all three of the cell lines. As expected, control FRhK-4 cells supported low levels of growth of HAV8Y-Bsd but did not support the growth of wt HM-175 HAV and HAV.WT-Bsd, which do not contain cell culture-adapting mutations. HAV.WT-Bsd and wt HM-175 HAV barely grew in parental Huh7 cells whereas HAV8Y-Bsd grew approximately 1 log₁₀, which showed that the main cell culture-adapting mutation at position 3889 had a marginal effect in these cells compared to FRhK-4 cells. The wt HAV viruses grew 10-fold better in Huh7-A-I cells than in the parental Huh7 cells indicating that the cured cells are highly permissive for wt HAV growth. HAV8Y-Bsd also grew 1 log₁₀ more in Huh7-A-I cells than the two wt HAV that do not contain the 3889 mutation. Consequently, the 2B/A16V change played a minor role in the susceptibility of Huh7 and Huh7-A-I cells to wt HAV infection compared to the major role it played in the susceptibility of FRhK-4 cells, where it is absolutely required for viral growth. Interestingly, the insertion of the bsd gene into the wt HAV genome did not have a mayor effect in viral growth since HAV.WT-Bsd and wt HM-175 HAV grew similarly in Huh7 and Huh7A-I cells. Nucleotide sequence analysis of the 5′ NCR and 2B-2C genes (FIG. 5B) was performed to verify that the genotype of the viruses produced in the different cell lines resembled the input virus. RT-PCR fragments amplified from genomic RNA extracted from virions confirmed that wt HM-175 HAV and HAV.WT-Bsd did not contain cell culture-adapting mutations, HAV8Y-Bsd contained a cell culture-adapting mutations at nucleotide 3889, and HAV/7 had a cluster of 6 cell culture-adapting mutations in the 2B-2C genes and another cluster of mutations in the 5′-NTR. Consequently, the genotypes of these viruses correlated with their phenotypes in FRhK-4 cells.

Various items of the patent and scientific periodical literature are cited herein. Each of these items is hereby incorporated by reference in its entirety and for all purposes by such citation.

### REFERENCES

Beard, M. R., L. Cohen, S. M. Lemon, and A. Martin. 2001. Characterization of Recombinant Hepatitis A Virus Genomes Containing Exogenous Sequences at the 2A/2B Junction. J. Virol. 75:1414-1426.
Bergmann, E. M., S. C. Mosimann, M. M. Chernaia, B. A. Malcolm, and M. N. James. 1997. The refined crystal structure of the 3C gene product from hepatitis A virus: specific proteinase activity and RNA recognition. J Virol 71:2436-48.
Borman, A. M., Y. M. Michel, and K. M. Kean. 2001. Detailed analysis of the requirements of hepatitis A virus internal ribosome entry segment for the eukaryotic initiation factor complex eIF4F. J Virol 75:7864-71.
Brack, K., W. Frings, A. Dotzauer, and A. Vallbracht. 1998. A cytopathogenic, apoptosis-inducing variant of hepatitis A virus. J Virol 72:3370-6.
Cohen, J. I., B. Rosenblum, S. M. Feinstone, J. Ticehurst, and R. H. Purcell. 1989. Attenuation and cell culture adaptation of hepatitis A virus (HAV): a genetic analysis with HAV cDNA. J. Virol. 63:5364-5370.
Cohen, J. I., B. Rosenblum, J. R. Ticehurst, R. J. Daemer, S. M. Feinstone, and R. H. Purcell. 1987. Complete nucleotide sequence of an attenuated hepatitis A virus: comparison with wild-type virus. Proc Natl Acad Sci USA 84:2497-501.
Cohen, J. I., J. R. Ticehurst, S. M. Feinstone, B. Rosenblum, and R. H. Purcell. 1987. Hepatitis A virus cDNA and its RNA transcripts are infectious in cell culture. J Virol 61:3035-9.
Cohen, J. I., J. R. Ticehurst, R. H. Purcell, A. Buckler-White, and B. M. Baroudy. 1987. Complete nucleotide sequence of wild-type hepatitis A virus: comparison with different strains of hepatitis A virus and other picornaviruses. J Virol 61:50-9.
Crance, J. M., F. Leveque, S. Chousterman, A. Jouan, C. Trepo, and R. Deloince. 1995. Antiviral activity of recombinant interferon-alpha on hepatitis A virus replication in human liver cells. Antiviral Res 28:69-80.
Daemer, R. J., S. M. Feinstone, I. D. Gust, and R. H. Purcell. 1981. Propagation of human hepatitis A virus in African green monkey kidney cell culture: primary isolation and serial passage. Infect Immun 32:388-93.
Emerson, S. U., Y. K. Huang, C. McRill, M. Lewis, and R. H. Purcell. 1992. Mutations in both the 2B and 2C genes of hepatitis A virus are involved in adaptation to growth in cell culture. J Virol 66:650-4.
Emerson, S. U., Y. K. Huang, C. McRill, M. Lewis, M. Shapiro, W. T. London, and R. H. Purcell. 1992. Molecular basis of virulence and growth of hepatitis A virus in cell culture. Vaccine 10:S36-9.
Emerson, S. U., Y. K. Huang, and R. H. Purcell. 1993. 2B and 2C mutations are essential but mutations throughout the genome of HAV contribute to adaptation to cell culture. Virology 194:475-80.
Emerson, S. U., M. Lewis, S. Govindarajan, M. Shapiro, T. Moskal, and R. H. Purcell. 1992. cDNA clone of hepatitis A virus encoding a virulent virus: induction of viral hepatitis by direct nucleic acid transfection of marmosets. J Virol 66:6649-54.
Emerson, S. U., C. McRill, B. Rosenblum, S. Feinstone, and R. H. Purcell. 1991. Mutations responsible for adaptation of hepatitis A virus to efficient growth in cell culture. J Virol 65:4882-6.
Fiore, A. E. 2004. Hepatitis A transmitted by food. Clin Infect Dis 38:705-15.
Goswami, B. B., M. Kulka, D. Ngo, and T. A. Cebula. 2004. Apoptosis induced by a cytopathic hepatitis A virus is dependent on caspase activation following ribosomal RNA degradation but occurs in the absence of 2′-5′ oligoadenylate synthetase. Antiviral Res 63:153-66.
Graff, J., and S. U. Emerson. 2003. Importance of amino acid 216 in nonstructural protein 2B for replication of hepatitis A virus in cell culture and in vivo. J Med Virol 71:7-17.
Graff, J., A. Normann, S. M. Feinstone, and B. Flehmig. 1994. Nucleotide sequence of wild-type hepatitis A virus GBM in comparison with two cell culture-adapted variants. J Virol 68:548-54.
Graff, J., A. Normann, and B. Flehmig. 1997. Influence of the 5′ noncoding region of hepatitis A virus strain GBM on its growth in different cell lines [published erratum appears in J Gen Virol 1998 January; 79(Pt 1):211]. J Gen Virol 78:1841-9.
Graff, J., O. C. Richards, K. M. Swiderek, M. T. Davis, F. Rusnak, S. A. Harmon, X. Y. Jia, D. F. Summers, and E. Ehrenfeld. 1999. Hepatitis A virus capsid protein VP1 has a heterogeneous C terminus. J Virol 73:6015-23.
Harmon, S. A., W. Updike, X. Y. Jia, D. F. Summers, and E. Ehrenfeld. 1992. Polyprotein processing in cis and in trans by hepatitis A virus 3C protease cloned and expressed in Escherichia coli. J Virol 66:5242-7.
Hollinger, F. B., and S. U. Emerson. 2001. Hepatitis A virus, p. 799-840. In D. M. Knipe and P. M. Howley (ed.), Fields Virology, Fourth ed, vol. 1. Lippincott Williams & Wilkins, Philadelphia.
Jansen, R. W., J. E. Newbold, and S. M. Lemon. 1988. Complete nucleotide sequence of a cell culture-adapted variant of hepatitis A virus: comparison with wild-type virus with restricted capacity for in vitro replication. Virology 163:299-307.
Jia, X. Y., D. F. Summers, and E. Ehrenfeld. 1993. Primary cleavage of the HAV capsid protein precursor in the middle of the proposed 2A coding region. Virology 193:515-9.
Kulka, M., A. Chen, D. Ngo, S. S. Bhattacharya, T. A. Cebula, and B. B. Goswami. 2003. The cytopathic 18f strain of Hepatitis A virus induces RNA degradation in FrhK4 cells. Arch Virol 148:1275-300.
Kusov, Y. Y., W. Sommergruber, M. Schreiber, and V. Gauss-Muller. 1992. Intermolecular cleavage of hepatitis A virus (HAV) precursor protein P1-P2 by recombinant HAV proteinase 3C. J Virol 66:6794-6.
Lemon, S. M., P. C. Murphy, P. A. Shields, L. H. Ping, S. M. Feinstone, T. Cromeans, and R. W. Jansen. 1991. Antigenic and genetic variation in cytopathic hepatitis A virus variants arising during persistent infection: evidence for genetic recombination. J Virol 65:2056-65.
Lohmann, V., F. Korner, J. Koch, U. Herian, L. Theilmann, and R. Bartenschlager. 1999. Replication of subgenomic hepatitis C virus RNAs in a hepatoma cell line. Science 285:110-3.
MacGregor, A., M. Kornitschuk, J. G. Hurrell, N. I. Lehmann, A. G. Coulepis, S. A. Locarnini, and I. D. Gust. 1983. Monoclonal antibodies against hepatitis A virus. Clin Microbiol 18:1237-43.
Martin, A., N. Escriou, S. F. Chao, M. Girard, S. M. Lemon, and C. Wychowski. 1995. Identification and site-directed mutagenesis of the primary (2A/2B) cleavage site of the hepatitis A virus polyprotein: functional impact on the infectivity of HAV RNA transcripts. Virology 213:213-22.
Nasser, A. M., and T. G. Metcalf. 1987. Production of cytopathology in FRhK-4 cells by BS-C-1-passaged hepatitis A virus. Appl Environ Microbiol 53:2967-71.
Pagano, J. S., and A. Vaheri. 1965. Enhancement of infectivity of poliovirus RNA with diethylaminoethyl-dextran (DEAE-D). Arch Gesamte Virusforsch 17:456-64.
Reed, L. J., and H. Muench. 1938. A simple method of estimating fifty percent end points. Am. J. Hyg. 27:493-497.
Sambrook, J., E. F. Fritsch, and T. Maniatis. 1989. Molecular cloning: A laboratory manual, 2nd ed. Cold Spring Harbor Laboratory, Cold Spring Harbor, N.Y.
Schultheiss, T., W. Sommergruber, Y. Kusov, and V. Gauss-Muller. 1995. Cleavage specificity of purified recombinant hepatitis A virus 3C proteinase on natural substrates. J Virol 69:1727-33.
Tesar, M., S. A. Harmon, D. F. Summers, and E. Ehrenfeld. 1992. Hepatitis A virus polyprotein synthesis initiates from two alternative AUG codons. Virology 186:609-18.
Tesar, M., X. Y. Jia, D. F. Summers, and E. Ehrenfeld. 1993. Analysis of a potential myristoylation site in hepatitis A virus capsid protein VP4. Virology 194:616-26.
Totsuka, A., and Y. Moritsugu. 1994. Hepatitis A vaccine development in Japan, p. 509-513. In K Nishioka, H. Suzuki, S. Mishiro, and T. Oda (ed.), Viral hepatitis and liver disease. Springer-Verlag, Tokyo.
Zhang, H., S. F. Chao, L. H. Ping, K Grace, B. Clarke, and S. M. Lemon. 1995. An infectious cDNA clone of a cytopathic hepatitis A virus: genomic regions associated with rapid replication and cytopathic effect. Virology 212:686-97.
Zhang, Y., and G. G. Kaplan. 1998. Characterization of replication-competent hepatitis A virus constructs containing insertions at the N terminus of the polyprotein. J Virol 72:349-57.
Weitz, M., B. M. Baroudy, et al. (1986). Detection of a genome-linked protein (VPg) of hepatitis A virus and its comparison with other picornaviral VPgs.” J Virol 60(1): 124-30.
Flehmig, B., A. Vallbracht, et al. (1981). Hepatitis A virus in cell culture. III. Propagation of hepatitis A virus in human embryo kidney cells and human embryo fibroblast strains. Med Microbiol Immunol 170(2): 83-9.
Binn, L. N., S. M. Lemon, et al. (1984). Primary isolation and serial passage of hepatitis A virus strains in primate cell cultures. J Clin Microbiol 20(1): 28-33.
Siegl, G., J. deChastonay, et al. (1984). Propagation and assay of hepatitis A virus in vitro. J Virol Methods 9(1): 53-67.
Ross, B. C., B. N. Anderson, et al. (1986). Molecular cloning of cDNA from hepatitis A virus strain HM-175 after multiple passages in vivo and in vitro. J Gen Virol 67(Pt 8): 1741-4.
Graff, J., C. Kasang, et al. (1994). Mutational events in consecutive passages of hepatitis A virus strain GBM during cell culture adaptation. Virology 204(1): 60-8.
Funkhouser, A. W., R. H. Purcell, et al. (1994). Attenuated hepatitis A virus: genetic determinants of adaptation to growth in MRC-5 cells. J Virol 68(1): 148-57.
Day, S. P., P. Murphy, et al. (1992). Mutations within the 5′ nontranslated region of hepatitis A virus RNA which enhance replication in BS-C-1 cells. J. Virol. 66: 6533-6540.
Funkhouser, A. W. et al. 1999. Hepatitis A Virus translation is rate-limit for virus replication in MRC-5 cells. Virology. 254:268-278.
Blight, K. J. et al. 2002. Highly permissive cell lines for subgenomic and genomic hepatitis C virus RNA replication. J. Virol. 76:13001-13014.
Tedeschi, V. et al. 1993. Partial characterization of Hepatitis A Viruses from three intermediate passage levels of a series resulting in adaptation to growth in cell culture and attenuation of virulence. J. Med. Virol. 39:16-22.
Amicone, L. et al. 1997. Transgenic expression in the liver of truncated Met blocks apoptosis and permits immortalization of hepatocytes. Embo J 16(3): 495-503.

1. A recombinant Hepatitis A Virus nucleic acid comprising:
i) a nucleotide sequence selected from the group consisting of
a) SEQ ID NO: 1,
b) the nucleotide sequence of SEQ ID NO: 1 at nucleotide 3889 in which a codon encodes valine at amino acid 216 of the 2B protein and
c) SEQ ID NO: 1 having a mutation at nucleotide 4087 in which a codon encodes methionine and a mutation at nucleotide 4222 in which a codon encodes serine,
d) SEQ ID NO: 1 having a mutation at nucleotide 3889 in which a codon encodes valine at amino acid 216 of the 2B protein and a mutation at nucleotide 4087 in which a codon encodes methionine, and
e) SEQ ID NO: 1 having a mutation at nucleotide 3889 in which a codon encodes valine at amino acid 216 of the 2B protein and a mutation at nucleotide 4222 in which a codon encodes serine;
ii) a nucleotide sequence representing at least one unique restriction enzyme site located between nucleotides encoding 3C^{pro} cleavage sites in the genome of the Hepatitis A Virus.

2. The recombinant Hepatitis A Virus nucleic acid of claim 1, in which the 3C^{pro} cleavage sites are in turn located at the junction of the 2A and 2B genes of the recombinant Hepatitis A Virus.

3. A DNA expression vector comprising a DNA recombinant Hepatitis A Virus nucleic acid of claim 1 operatively linked to a promoter for transcription of a genomic RNA of the recombinant Hepatitis A Virus.

4. The expression vector of claim 3, in which the promoter is one suitable for in vitro transcription of the viral genomic RNA.

5. A recombinant Hepatitis A Virus nucleic acid comprising:
i) a nucleotide sequence selected from the group consisting of
a) SEQ ID NO: 1,
b) the nucleotide sequence of SEQ ID NO: 1 at nucleotide 3889 in which a codon encodes valine at amino acid 216 of the 2B protein and
c) SEQ ID NO: 1 having a mutation at nucleotide 4087 in which a codon encodes methionine and a mutation at nucleotide 4222 in which a codon encodes serine,
d) SEQ ID NO: 1 having a mutation at nucleotide 3889 in which a codon encodes valine at amino acid 216 of the 2B protein and a mutation at nucleotide 4087 in which a codon encodes methionine, and
e) SEQ ID NO: 1 having a mutation at nucleotide 3889 in which a codon encodes valine at amino acid 216 of the 2B protein and a mutation at nucleotide 4222 in which a codon encodes serine; and
ii) a nucleic acid encoding a protein conferring a selectable or screenable phenotype upon a cell that expresses said protein.

6. The recombinant Hepatitis A Virus nucleic acid of claim 5, in which the nucleic acid ii) is located between nucleotides encoding 3C^{pro} cleavage sites in the genome of the Hepatitis A Virus.

7. The recombinant Hepatitis A Virus of claim 6, in which the 3C^{pro} cleavage sites are in turn located at the junction of the 2A and 2B genes of the recombinant Hepatitis A Virus.

8. A DNA expression vector comprising a DNA recombinant Hepatitis A Virus nucleic acid of claim 5 operatively linked to a promoter for transcription of a genomic RNA of the recombinant Hepatitis A Virus.

9. The expression vector of claim 8, in which the promoter is one suitable for in vitro transcription of the viral genomic RNA.

10. The recombinant Hepatitis A Virus nucleic acid of claim 5, wherein the selectable or screenable phenotype is resistance to an antibiotic that is effective against cultured mammalian cells and inhibits protein translation in the cultured mammalian cells.

11. A recombinant Hepatitis A Virus nucleic acid comprising a nucleotide sequence of a genome of said Hepatitis A Virus and a nucleotide sequence providing resistance to an antibiotic that inhibits translation in a mammalian cell or that provides resistance to a drug that induces apoptosis in a mammalian cell.

12. (canceled)

13. A recombinant Hepatitis A Virus nucleic acid comprising
i) a nucleotide sequence representing at least one unique restriction enzyme site located between nucleotides encoding protease 3C^{pro} cleavage sites; and
ii) a nucleotide sequence located between nucleotides encoding protease 3C^{pro} cleavage sites providing resistance to an antibiotic that is effective against cultured mammalian cells and inhibits protein translation or promotes apoptosis in the cultured mammalian cells;
wherein cells that replicate the recombinant Hepatitis A Virus nucleic acid can be selected by the antibiotic resistance or apoptotic phenotype.

14. A recombinant Hepatitis A Virus nucleic acid comprising:
i) a nucleotide sequence selected from the group consisting of
a) SEQ ID NO: 1,
b) the nucleotide sequence of SEQ ID NO: 1 at nucleotide 3889 in which a codon encodes valine at amino acid 216 of the 2B protein and
c) SEQ ID NO: 1 having a mutation at nucleotide 4087 in which a codon encodes methionine and a mutation at nucleotide 4222 in which a codon encodes serine,
d) SEQ ID NO: 1 having a mutation at nucleotide 3889 in which a codon encodes valine at amino acid 216 of the 2B protein and a mutation at nucleotide 4087 in which a codon encodes methionine, and
e) SEQ ID NO: 1 having a mutation at nucleotide 3889 in which a codon encodes valine at amino acid 216 of the 2B protein and a mutation at nucleotide 4222 in which a codon encodes serine; and
ii) a nucleotide sequence representing at least one heterologous nucleotide sequence located between nucleotides encoding protease 3C^{pro} cleavage sites in the Hepatitis A Virus genome.

15. The Hepatitis A Virus nucleic acid of claim 14, in which the 3C^{pro} cleavage sites are in turn located between the 2A and 2B genes of the Hepatitis A Virus.

16. A Hepatitis A Virus particle comprising a nucleic acid of claim 14.

17. A method for selecting a cell permissive for replication of Hepatitis A Virus comprising:
i) transfecting cultured cells with the recombinant Hepatitis A Virus nucleic acid of claim 5; and
ii) selecting or screening the transfected cells for the phenotype conferred by the recombinant Hepatitis A Virus;
iii) wherein a cell exhibiting the selected or screened phenotype is deemed to be permissive for growth and replication of Hepatitis A Virus.

18. A method for selecting a cell permissive for replication of Hepatitis A Virus comprising:
i) transfecting cultured cells with the recombinant Hepatitis A Virus nucleic acid of claim 11; and
ii) selecting or screening the transfected cells for the phenotype conferred by the recombinant Hepatitis A Virus;
iii) wherein a cell exhibiting resistance to the antibiotic or to apoptosis is deemed to be permissive for growth and replication of Hepatitis A Virus.

19. The method of claim 17 that further comprises curing the selected cell of the Hepatitis A Virus nucleic acid.

20. The method of claim 18 that further comprises curing the selected cell of the Hepatitis A Virus nucleic acid.

21. The method of claim 17, further comprising testing the cell for growth of wild-type Hepatitis A Virus or of an attenuated Hepatitis A Virus.

22. The method of claim 18, further comprising testing the cell for growth of wild-type Hepatitis A Virus or of an attenuated Hepatitis A Virus.

23. The method of claim 19, further comprising testing the cell for growth of wild-type Hepatitis A Virus or of an attenuated Hepatitis A Virus.

24. The method of claim 20, further comprising testing the cell for growth of wild-type Hepatitis A Virus or of an attenuated Hepatitis A Virus.

25. The method of claim 17, wherein the phenotype is resistance to an antibiotic that is effective against cultured mammalian cells.

26. A mammalian cell line comprising cells that have been selected by the method of claim 17.

27. A mammalian cell line comprising cells that have been selected by the method of claim 18.

28. A mammalian cell line comprising cells that have been selected by the method of claim 17 and are permissive for growth of wild-type HAV.

29. A mammalian cell line comprising cells that have been selected by the method of claim 18 and are permissive for growth of wild-type HAV.

30. A mammalian cell line comprising Huh7 cells that have been transfected with a recombinant Hepatitis A Virus nucleic acid comprising a nucleic acid encoding a protein conferring a selectable or screenable phenotype upon a cell that expresses said protein that is located between nucleotides encoding 3C^{pro} cleavage sites in the genome of said Hepatitis A Virus and then subsequently cured of the recombinant Hepatitis A Virus, said cells being permissive for replication of Hepatitis A Virus.

31. The cell line of claim 30, in which the recombinant Hepatitis A Virus comprises a codon encoding valine at amino acid 216 of the 2B protein.

32. The cell line of claim 30, in which the screenable or selectable phenotype is resistance to an antibiotic that inhibits protein translation in mammalian cells.

33. The cell line of claim 32, in which the antibiotic is blasticidin, puromycin or a puromycin derivative.

34. A human hepatoma cell line Huh7-A-I deposited at the American Type Culture Collection as PTA-6773.

35. A method for producing a Hepatitis A Virus comprising infecting a cell with said Hepatitis A Virus particles, or transfecting a cell with a nucleic acid representing the genome of a Hepatitis A Virus, culturing the infected or transfected cell to provide for replication of the Hepatitis A Virus, and separating particles of the Hepatitis A Virus from the cultured cells,
wherein the cell is one from the cell line of claim 26.

36. A method for assaying a sample for infectious Hepatitis A Virus comprising contacting the sample with cells from a cell line of claim 26, culturing the cells, and then determining the presence of Hepatitis A Virus in the sample by a method selecting from the group consisting of:
i) titering the virus present in the cultured cells by contacting a sample of a supernatant of the culture with mammalian cells that may be infected by Hepatitis A Virus and counting cytopathic plaques;
ii) performing a polymerase chain reaction using primers specific for Hepatitis A Virus nucleic acids and a nucleic acid sample prepared from cells of the culture as a template;
and iii) assaying for the presence of at least one protein specific to Hepatitis A Virus by an immunoassay method.

37. A method for producing a Hepatitis A Virus nucleic acid comprising infecting a cell with Hepatitis A Virus particles, or transfecting a cell with a nucleic acid representing the genome of a Hepatitis A Virus, culturing the infected or transfected cell to provide for replication of the Hepatitis A Virus, separating particles of the Hepatitis A Virus from the cultured cells, and purifying Hepatitis A Virus nucleic acids from the separated particles,
wherein the cell is one from the cell line of claim 26.

38. A method for producing a Hepatitis A Virus nucleic acid comprising infecting a Huh7 cell or a cell of a hepatoma cell line with Hepatitis A Virus particles, or transfecting said cell with a nucleic acid representing the genome of a Hepatitis A Virus, culturing the infected or transfected cell to provide for replication of the Hepatitis A Virus, separating particles of the Hepatitis A Virus from the cultured cells, and purifying Hepatitis A Virus nucleic acids from the separated particles.

39. The method of claim 37, in which the Hepatitis A Virus is one comprising
a nucleotide sequence representing at least one heterologous nucleotide sequence located between nucleotides encoding protease 3C^{pro} cleavage sites in the genome of the recombinant Hepatitis A Virus.

40. A method for using a recombinant Hepatitis A Virus comprising a selectable marker gene to identify cellular factors that allow growth of wild-type Hepatitis A Virus in cells containing such cellular factors, comprising:
I) selecting from a collection of cells that are non-permissive for replication and growth of Hepatitis A Virus, said collection of cells having been transformed with a library of nucleic acids made from a hepatoma cell line in an expression vector, one or more cells that express a selectable or screenable phenotype, said selectable or screenable phenotype being conferred by infection with a virus comprising a nucleic acid, or being conferred by transfection with a nucleic acid, said nucleic acid comprising;
i) a nucleotide sequence of SEQ ID NO: 1, and
ii) a nucleic acid encoding a protein conferring a selectable or screenable phenotype upon a cell that expresses said protein that is located between nucleotides encoding 3C^{pro} cleavage sites in the genome of the wild type Hepatitis A Virus; and
II) determining the nucleotide sequence of the nucleic acid present in the expression vector of the selected cell(s).
